(19) United States
(12) Patent Application Publication
(10) Pub. No.: US 2025/0170056 A1
(43) Pub. Date: May 29, 2025

(54) Soft Chew Tablets Suitable For Antacids

(71) Applicant: Fertin Pharma A/S, Vejle (DK)

(72) Inventors: Helle Wittorff, Vejle Ost (DK); Jane Skov Grabow Jensen, Østbirk (DK)

(73) Assignee: Fertin Pharma A/S, Vejle (DK)

(21) Appl. No.: 18/958,419

(22) Filed: Nov. 25, 2024

(30) Foreign Application Priority Data
Nov. 25, 2023 (DK) PA202330347

(51) Int. Cl. A61K 9/00; A61K 33/10; A61K 47/10; A61K 47/26; A61K 47/36; A61K 47/42

(52) U.S. Cl. CPC A61K 9/0056; A61K 33/10; A61K 47/10; A61K 47/26; A61K 47/36; A61K 47/42

(57) ABSTRACT

A coated soft chew tablet includes a core in an amount of 60 to 99% by weight of the soft chew tablet. The core constituting a coherent uncooked mass includes one or more water soluble fibers in an amount of 10 to 45% by weight of the core, one or more powdered monosaccharides and/or disaccharides in an amount of 10 to 45% by weight of the core, one or more texturizers in an amount of 0.1 to 15% by weight of the core, one or more active ingredients in an amount of 1 to 60% by weight of the core, and one or more solid sugar syrups in an amount up to 20% by weight of the core; and an outer coating surrounding the core in an amount of 1 to 40% by weight of the soft chew tablet.

### FIELD OF INVENTION

The present invention relates to soft chew tablets with active ingredients, such as pharmaceutical or nutraceutical active ingredients. The soft chew tablets are particularly suitable for antacids with gastrointestinal activity, such as calcium carbonate.

### BACKGROUND

Various attempts have been made to apply soft chew tablets used in the confectionery industry as delivery vehicles for pharmaceutical active ingredients. While soft chew tablets are generally attractive for consumers within the confectionery segment, various issues arise if active ingredients are to be formulated into these products.

Traditionally, soft chew tablets are made in a manufacturing process that involves recrystallization of sugars and other ingredients and involving extensive heat treatment during processing. Although the texture of such products becomes consumer friendly, high temperatures may be challenging to heat sensitive active ingredients. Also, melting of ingredients may impact release characteristics in the products as well as the required load level of such ingredients in the products.

Another challenge of heat treatment in soft chew tablets and especially cooking during the process is that the texture of the products becomes unsuitable for delivery of certain active ingredients, such as calcium carbonate for alleviation or treatment of gastrointestinal reflux. The need for delivery of large doses of active ingredients may be counteracted by a dense and compacted structure of the soft chew tablets. Additionally, high loads of active ingredients may in itself counteract the intended texture of such products.

In recent years, variations of the manufacturing process of soft chew tablets have been suggested for active ingredients, processes that involve less heat transfer to the ingredients during the manufacturing process. However, various challenges still apply for these processes, such as undesirable texture of the products as well as a high degree of stickiness of the products to surfaces.

Hence, there is a need in the prior art for improved delivery vehicles that solve the above-referenced challenges and problems of the prior art. In particular, there is a need in the prior art for new soft chew tablet delivery vehicles that support appropriate delivery of active ingredients, such as calcium carbonate, combined with beneficial sensorial properties, including improved mouthfeel and texture.

### SUMMARY

Accordingly, in a first aspect of the invention, there is provided a coated soft chew tablet, the soft chew tablet comprising: a core in an amount of 60 to 99% by weight of the soft chew tablet, the core constituting a coherent uncooked mass comprising: one or more water soluble fibers in an amount of 10 to 45% by weight of the core, one or more powdered monosaccharides and/or disaccharides in an amount of 10 to 45% by weight of the core, one or more texturizers in an amount of 0.1 to 15% by weight of the core, one or more active ingredients in an amount of 1 to 60% by weight of the core, and one or more solid sugar syrups in an amount up to 20% by weight of the core; and an outer coating surrounding the core in an amount of 1 to 40% by weight of the soft chew tablet.

In a second aspect, there is provided a coated soft chew tablet suitable for actives, the soft chew tablet comprising: a core in an amount of 60 to 95% by weight of the soft chew tablet and an outer hard coating surrounding the core in an amount of 5 to 40% by weight of the soft chew tablet, the core constituting a coherent uncooked mass comprising: one or more water soluble fibers in an amount of 10 to 45% by weight of the core, one or more powdered monosaccharides and/or disaccharides in an amount of 10 to 45% by weight of the core, one or more texturizers in an amount of 0.1 to 15% by weight of the core, one or more active ingredients in an amount of 1 to 60% by weight of the core, and one or more solid sugar syrups in an amount up to 20% by weight of the core.

In a third aspect, there is provided a coated soft chew tablet suitable for calcium carbonate, the soft chew tablet comprising: a core in an amount of 60 to 95% by weight of the soft chew tablet and an outer hard coating surrounding the core in an amount of 5 to 40% by weight of the soft chew tablet, the core constituting a coherent uncooked mass comprising: one or more water soluble fibers in an amount of 10 to 45% by weight of the core, one or more powdered monosaccharides and/or disaccharides in an amount of 10 to 45% by weight of the core, one or more texturizers in an amount of 0.1 to 15% by weight of the core, calcium carbonate in an amount of 10 to 60% by weight of the core, and one or more solid sugar syrups in an amount up to 20% by weight of the core.

In a fourth aspect, there is provided a coated soft chew tablet suitable for antacids, the soft chew tablet comprising: a core in an amount of 60 to 95% by weight of the soft chew tablet and an outer hard coating surrounding the core in an amount of 5 to 40% by weight of the soft chew tablet, the core constituting a coherent uncooked mass comprising: one or more water soluble fibers in an amount of 10 to 45% by weight of the core, one or more powdered monosaccharides and/or disaccharides in an amount of 10 to 45% by weight of the core, one or more texturizers in an amount of 0.1 to 15% by weight of the core, one or more antacids in an amount of 10 to 60% by weight of the core, and one or more solid sugar syrups in an amount up to 20% by weight of the core. The various aspects of the invention may be suitable for antacids, such as calcium carbonate.

Generally, the present invention solves various of the challenges of the prior art and may provide further advantages compared to the prior art in terms of improved mouthfeel, texture as well as improved delivery of active ingredients. An important advantage is that the stickiness of the product may be reduced or even avoided by the present invention, which means that handling and convenience may be greatly improved. Another important advantage is that challenges in terms of moisture balance in the product may be greatly improved.

Additionally, it was a surprise to the inventors that a coating surrounding the core of the present invention could help avoiding too much hardening over time during storage. While soft coatings and film coatings contributed to a significant degree, particularly a hard coating was beneficial. The hardening of the core during storage was regularly measured with a texture analyzer and it could be seen that hardening was more pronounced without a coating. This was a surprise to the inventors.

In some embodiments, the outer coating, such as outer hard coating, constitutes 2 to 40% by weight of the soft chew tablet. In some embodiments, the outer coating, such as outer hard coating, constitutes 3 to 40% by weight of the soft chew tablet. the outer coating constitutes 5 to 40% by weight of the soft chew tablet. In some embodiments, the outer coating, such as outer hard coating, constitutes 10 to 35% by weight of the soft chew tablet. In some embodiments, the outer hard coating, such as outer hard coating, constitutes 15 to 30% by weight of the soft chew tablet. In some embodiments, the outer hard coating, such as outer hard coating, constitutes 20 to 35% by weight of the soft chew tablet. In some embodiments, the outer hard coating, such as outer hard coating, constitutes 20 to 30% by weight of the soft chew tablet.

In some embodiments, the outer coating comprises a suspension of sugar applied to the core in a pan coating process. In some embodiments, the outer coating comprises a suspension of sugar alcohols applied to the core in a pan coating process.

In some embodiments, the outer coating comprises a suspension of sugar applied during multiple cycles to the core in a pan coating process, such as during 3 to 80 cycles, to provide a hard coating comprising multiples layers. In some embodiments, the outer coating comprises a suspension of sugar alcohols applied during multiple cycles to the core in a pan coating process, such as during 3 to 80 cycles, to provide a hard coating comprising multiples layers.

In the present context, the term “hard coating” is used in the conventional meaning of that term. The hard coating provides a crunchy layer, which is appreciated by the consumer and protects the cores of the soft chew tablets as seen according to the invention. In a typical process of providing the cores with a protective sugar coating, the cores are successively treated in suitable coating equipment with aqueous solutions of crystallizable sugar such as sucrose or dextrose, which, depending on the stage of coating reached, may contain other functional ingredients, e.g., fillers, binding agents, flavors, colors, etc. In the present context, “functional ingredients” is not intended to cover active ingredients, such as “nutraceutical ingredients” or the like.

In a typical hard coating process as it will be described in detail in the following, a suspension containing crystallizable sugar and/or polyol is applied onto the cores and the water it contains is evaporated off by blowing with air. This cycle must be repeated several times, typically 3 to 80 times, in order to reach the swelling required. The term “swelling” refers to the increase in weight or thickness of the products, as considered at the end of the coating operation by comparison with the beginning, and in relation to the final weight or thickness of the coated products.

In some embodiments, the outer coating comprises one or more fillers, binding agents, colors, or flavors.

In some embodiments, the outer coating comprises binding agents, such as acacia or gelatine. In some embodiments, the outer hard coating comprises binding agents including acacia. In some embodiments, the outer hard coating comprises binding agents including gelatine.

In some embodiments, the outer coating comprises binding agents in an amount of 1 to 10% by weight of the coating. In some embodiments, the outer coating comprises binding agents in an amount of 2 to 10% by weight of the coating. In some embodiments, the outer coating comprises binding agents in an amount of 2 to 8% by weight of the coating. In some embodiments, the outer coating comprises binding agents in an amount of 3 to 8% by weight of the coating. In some embodiments, the outer coating comprises binding agents in an amount of 4 to 7% by weight of the coating. The amount of binding agents are based on dry weight.

In some embodiments, the outer coating comprises sugar selected from the group consisting of sucrose, glucose, fructose, galactose, and any combination thereof. In some embodiments, the outer coating comprises sucrose. In some embodiments, the outer coating comprises glucose. In some embodiments, the outer coating comprises fructose. In some embodiments, the outer coating comprises galactose. In some embodiments, the outer coating comprises a combination of sugars. It is noted that “glucose” and “dextrose” is the same, hence “dextrose” may be present. In some embodiments, the outer coating is sucrose.

In some embodiments, the outer coating comprises sugar in an amount of 30 to 98% by weight of the coating, such as in an amount of 50 to 95% by weight of the coating. In some embodiments, the outer coating comprises sugar in an amount of 30 to 95% by weight of the coating, such as in an amount of 50 to 95% by weight of the coating. The percentages are based on dry weight.

In some embodiments, the outer coating comprises sugar in an amount of 30 to 98% by weight of the coating. In some embodiments, the outer coating comprises sugar in an amount of 40 to 98% by weight of the coating. In some embodiments, the outer coating comprises sugar in an amount of 50 to 98% by weight of the coating. In some embodiments, the outer coating comprises sugar in an amount of 50 to 95% by weight of the coating. In some embodiments, the outer coating comprises sugar in an amount of 60 to 95% by weight of the coating. In some embodiments, the outer coating comprises sugar in an amount of 70 to 95% by weight of the coating. In some embodiments, the outer coating comprises sugar in an amount of 80 to 95% by weight of the coating. In some embodiments, the outer coating comprises sugar in an amount of 90 to 95% by weight of the coating. The amounts as outlined above are also relevant for sugar alcohols, if present, in the coating.

In some embodiments, the outer coating, such as hard coating, constitutes a partial moisture barrier to ingredients in the core of the soft chew tablet. In the present context, “partial moisture barrier” is intended to mean that the coating, such as hard coating, does not necessarily invoke 100% barrier properties, while this is preferred, but may invoke less than 100% barrier properties. In some embodiments, “partial” implies more than 100% barrier properties calculated over 7 days of storage, excluding any moisture absorbed from the coating during processing. In some embodiments, “partial” implies more than 99% barrier properties. In some embodiments, “partial” implies more than 98% barrier properties. In some embodiments, “partial” implies more than 95% barrier properties. In some embodiments, “partial” implies more than 90% barrier properties. In some embodiments, “partial” implies more than 80% barrier properties. It is implied that “partial moisture barrier” may also be understood as “improvement of the moisture retention in the core”.

In some embodiments, the outer coating, such as hard coating, constitutes an anti-sticking barrier to ingredients in the core of the soft chew tablet. One of the challenges of the prior art is that the core may be sticky, which causes challenges in terms of handling of the products. It was a surprise to the inventors of the invention that the problems with stickiness could be solved by providing a coating, such as hard coating, to the cores according to the invention. Due to the relative wet conditions in the coating process, such as hard coating, it was expected that the core would be compromised by the coating process, including the texture. This was particularly expected for a hard coating. However, this was not seen, and the active ingredients appeared unaffected after the coating process. Hence, the coating process of the invention, such as hard coating, may be seen to solve several challenges, including the moisture balance as well as challenges with stickiness.

In some embodiments, the outer coating is a hard coating. In some embodiments, the outer coating is a coating selected from the group consisting of a film coating, a soft coating, and a chocolate coating.

In some embodiments, the soft chew tablet further comprises a sub-coat applied between the core and the outer coating, such as hard coating.

In some embodiments, the soft chew tablet further comprises a sub-coat applied between the core and the outer hard coating constituting 0.1 to 5% by weight of the soft chew tablet. In some embodiments, the soft chew tablet further comprises a sub-coat applied between the core and the outer hard coating constituting 0.1 to 4% by weight of the soft chew tablet. In some embodiments, the soft chew tablet further comprises a sub-coat applied between the core and the outer hard coating constituting 0.2 to 4% by weight of the soft chew tablet. In some embodiments, the soft chew tablet further comprises a sub-coat applied between the core and the outer hard coating constituting 0.2 to 3% by weight of the soft chew tablet. In some embodiments, the soft chew tablet further comprises a sub-coat applied between the core and the outer hard coating constituting 0.3 to 3% by weight of the soft chew tablet. In some embodiments, the soft chew tablet further comprises a sub-coat applied between the core and the outer hard coating constituting 0.5 to 3% by weight of the soft chew tablet. In some embodiments, the soft chew tablet further comprises a sub-coat applied between the core and the outer hard coating constituting 1 to 3% by weight of the soft chew tablet.

In some embodiments, the soft chew tablet further comprises a sub-coat applied between the core and the outer hard coating comprising maltodextrin. In some embodiments, the soft chew tablet further comprises a sub-coat applied between the core and the outer hard coating comprising glucose syrup. In some embodiments, the soft chew tablet further comprises a sub-coat applied between the core and the outer hard coating comprising shellac. Typically, the sub-coat (also known as pre-coat) is applied in a similar way as a film coating. In some embodiments, the sub-coat is a “film coating”. In some embodiments, another “film coating” may be applied on top of this film-coating to provided dual film coating, or multiple film coatings.

In further useful embodiments, the core of the soft chew tablets according to the invention is an element that is subjected to a film coating process and which therefore comprises one or more film-forming polymeric agents and optionally one or more auxiliary compounds, e.g., plasticizers, pigments and opacifiers. A film coating is a thin polymer-based coating applied to a core of any of the above forms. The thickness of such a coating is usually between 20 and 100 μm. Generally, the film coating is obtained by passing the cores through a spray zone with atomized droplets of the coating materials in a suitable aqueous or organic solvent vehicle, after which the material adhering to the cores is dried before the next portion of coating is received. This cycle is repeated until the coating is complete.

In the present context, suitable film-coating polymers include edible cellulose derivatives such as cellulose ethers including methylcellulose (MC), hydroxyethyl cellulose (HEC), hydroxypropyl cellulose (HPC) and hydroxypropyl methylcellulose (HPMC). Other useful film-coating agents are acrylic polymers and copolymers, e.g. methylacrylate aminoester copolymer or mixtures of cellulose derivatives and acrylic polymers. A particular group of film-coating polymers, also referred to as functional polymers are polymers that, in addition to its film-forming characteristics, confer a modified release performance with respect to active components of the cores. Such release modifying polymers include methylacrylate ester copolymers, ethylcellulose (EC) and enteric polymers designed to resist the acidic stomach environment. The latter group of polymers include: cellulose acetate phtalate (CAP), polyvinyl acetate phtalate (PVAP), shellac, metacrylic acid copolymers, cellulose acetate trimellitate (CAT) and HPMC. It will be appreciated that the outer film coating according to the present invention may comprise any combination of the above film-coating polymers as well as the components also mentioned elsewhere.

In some embodiments, the soft chew tablet further comprises a sub-coat applied between the core and the outer hard coating constituting a partial moisture barrier to ingredients in the core of the soft chew tablet.

In the present context, “partial moisture barrier” is intended to mean that the sub-coat does not necessarily invoke 100% barrier properties, while this is preferred, but may invoke less than 100% barrier properties. In some embodiments, “partial” implies more than 100% barrier properties calculated over 7 days of storage, excluding any moisture absorbed from the coating during processing. In some embodiments, “partial” implies more than 99% barrier properties. In some embodiments, “partial” implies more than 98% barrier properties. In some embodiments, “partial” implies more than 95% barrier properties. In some embodiments, “partial” implies more than 90% barrier properties. In some embodiments, “partial” implies more than 80% barrier properties. It is implied that “partial moisture barrier” may also be understood as “improvement of the moisture retention in the core”.

In some embodiments, the sub-coat constitutes an anti-sticking barrier to ingredients in the core of the soft chew tablet. One of the challenges of the prior art is that the core may be sticky, which causes challenges in terms of handling of the products. It was a surprise to the inventors of the invention that the problems with stickiness could be solved by providing a sub-coat to the cores according to the invention. Due to the relative wet conditions in the coating process, it was expected that the core would be compromised by the coating process, including the texture. However, this was not seen, and the active ingredients appeared unaffected after the coating process. Hence, the hard coating process of the invention may be seen to solve several challenges, including the moisture balance as well as challenges with stickiness.

In some embodiments, the soft chew tablet comprises the core in an amount of 60 to 99% by weight of the soft chew tablet. In some embodiments, the soft chew tablet comprises the core in an amount of 60 to 98% by weight of the soft chew tablet. In some embodiments, the soft chew tablet comprises the core in an amount of 60 to 95% by weight of the soft chew tablet. In some embodiments, the soft chew tablet comprises the core in an amount of 60 to 90% by weight of the soft chew tablet. In some embodiments, the soft chew tablet comprises the core in an amount of 70 to 95% by weight of the soft chew tablet. In some embodiments, the soft chew tablet comprises the core in an amount of 70 to 90% by weight of the soft chew tablet. In some embodiments, the soft chew tablet comprises the core in an amount of 60 to 90% by weight of the soft chew tablet. In some embodiments, the soft chew tablet comprises the core in an amount of 60 to 80% by weight of the soft chew tablet.

In some embodiments, the coherent uncooked mass has not been re-crystallized during formation of the core. In the present context, “re-crystallized” is intended to mean that crystalline ingredients applied in the process are not melted during the process and re-crystallized during or after cooling. Typically, processes involving cooking, such as by heating crystalline ingredients and other ingredients to 130-150 degree Celsius during processing, completely melts the mass of ingredients, which results in the ingredients being spun together in the mass. During or after cooling, the mass of spun ingredients re-crystallizes in such cooking process. In the present context, cooking is not invoked to the ingredients, and the mass of core of the soft chew tablets is “an uncooked mass”. On a microscopic level, the texture of a cooked mass that has been re-crystallized may be substantially different than the texture of the mass of the core in the present invention, while retaining the characteristics of a “soft chew tablet” on a macroscopic, perceived level. Such cooked mass is associated with various drawbacks, including challenges with inferior release of active ingredients, damaging of heat sensitive active ingredients, stickiness, etc.

A person skilled in the art would understand the intentional meaning with “soft chew tablets” in the present context. By the formulation presented to the instantly claimed “soft chew tablets”, the inventors managed to resemble “soft chew tablets” made by the aforementioned cooking process, but without the challenges associated with this process. The formulation provided according to the invention is associated with various advantages, such as the possibility to contain a substantial amount of active ingredients, including heat-sensitive active ingredients, which is not possible with a cooked mass for traditional soft chew tablets.

By resembling “soft chew tablets”, the intended meaning is that texture with chew resistance is obtained, just like for cooked “soft chew tablets”. The chew resistance is characterized by some degree of elasticity and may be comparable with “chewing gum” for the first initial chews (some degree of elasticity and chew resistance), contrary to a plastic chew without elasticity as seen for products like toffees. However, compared to chewing gum and the first initial chews in this product, the “soft chew tablets” is absent any insoluble chewing gum base. Another non-comparable product is a fondant (a sugar mass that offers no resistance or elasticity when chewing it). Chew resistance may be measured by a “texture analyzer” known to one skilled in the art or by sensorial evaluation by a test panel.

In some embodiments, the one or more powdered monosaccharides and/or disaccharides of the coherent uncooked mass has been preserved during formation of the core.

In some embodiments, the coherent uncooked mass is characterized by preserved crystallinity of the one or more powdered monosaccharides and/or disaccharides during formation of the core. In the present context, “preserved” means up to 100% preserved, but there may be parts of the crystalline ingredients applied in the process that may not be preserved, such as for instance up to 5% by the crystalline ingredients.

In some embodiments, the one or more powdered monosaccharides and/or disaccharides has retained at least 90% crystallinity during formation of the core, such as at least 95% crystallinity.

In some embodiments, the coherent uncooked mass is a coherent non-crystalline mass.

In some embodiments, the coherent uncooked mass is characterized by a texture resembling a paste or dough mass with remains of particles.

In some embodiments, the coherent uncooked mass is extruded. Typically, the mass is made in a batch process and formed into a rope that is cut into round-shaped pieces whereupon a coating is applied. The mass may be conveyed to an extruder during the process. As an alternative to a batch process, a continuous extrusion process may be applied.

In some embodiments, the coherent uncooked mass is not compressed or pressed, such as in a tableting machine. Such compression is usually applied to particular material and gives a product that is distant from the characteristic “soft chew tablets” according to the invention. In some embodiments, the coherent uncooked mass is not molded. In some embodiments, the coherent uncooked mass is not a deposited mass. These products are distant from the characteristic “soft chew tablets” according to the invention.

In some embodiments, the coherent uncooked mass is formed without melting the one or more powdered monosaccharides and/or disaccharides during formation of the core, such as with a temperature below 60 degree Celsius, such as with a temperature of 30 to 50 degree Celsius.

In some embodiments, the coherent uncooked mass is formed at a temperature below 60 degree Celsius. In some embodiments, the coherent uncooked mass is formed at a temperature of 20 to 50 degree Celsius. In some embodiments, the coherent uncooked mass is formed at a temperature of 20 to 40 degree Celsius. In some embodiments, the coherent uncooked mass is formed at a temperature of 30 to 40 degree Celsius. In some embodiments, the coherent uncooked mass is formed at a temperature of 40 to 50 degree Celsius.

One of the advantages of the present invention is that stability of active ingredients may be surprisingly improved. Compared to other products, such as gummies and products made by cooking, the soft chew tablet of the present invention may offer better stability to active ingredients. Also, compared to tablets with a higher water content, the instant soft chew tablets may be improved in terms of stability. In the present context, “stability” is intended to mean that the active ingredients or at least one active ingredient according to the invention are only degraded to an acceptable level or subject to bond breaking to an acceptable level over time, such as limiting side products over time. One of the consequences if stability is not preserved is that the one or more active ingredients may decrease in activity over time or be subject to side products that may seriously compromise the quality of the soft chew tablets, such as the texture, taste, mouthfeel, or the like.

In some embodiments, the coherent uncooked mass is an unspun mass of ingredients.

In some embodiments, the coherent uncooked mass is formed without high speed mixing of ingredients.

In some embodiments, the coherent uncooked mass is formed by low speed shear mixing of ingredients, such as by a Z-blade mixer which is also known as a sigma-blade mixer or a sigma-blade kneader mixer. In this process, the mass may be cooled after mixing and formed into a rope by means of extrusion.

In some embodiments, the coherent uncooked mass is formed by mixing in an extruder in a continuous process. In this process, the mass may be cooled in a final zone of the extruder after mixing and directly formed into a rope by the extruder.

In some embodiments, the one or more water soluble fibers is present in an amount of 15 to 45% by weight of the core. In some embodiments, the one or more water soluble fibers is present in an amount of 15 to 40% by weight of the core. In some embodiments, the one or more water soluble fibers is present in an amount of 20 to 40% by weight of the core. In some embodiments, the one or more water soluble fibers is present in an amount of 25 to 40% by weight of the core. In some embodiments, the one or more water soluble fibers is present in an amount of 20 to 35% by weight of the core. In some embodiments, the one or more water soluble fibers is present in an amount of 20 to 30% by weight of the core.

In some embodiments, the one or more water soluble fibers has a Dextrose Equivalent (DE) of 3 to 20. In some embodiments, the one or more water soluble fibers has a Dextrose Equivalent (DE) of 5 to 20. In some embodiments, the one or more water soluble fibers has a Dextrose Equivalent (DE) of 10 to 20. In some embodiments, the one or more water soluble fibers has a Dextrose Equivalent (DE) of 12 to 20. In some embodiments, the one or more water soluble fibers has a Dextrose Equivalent (DE) of 15 to 20.

Dextrose Equivalent is understood by a person skilled in the art and is applied in the present context as understood in the art. Starch has a DE of 0, while dextrose has a DE of 100.

In some embodiments, the one or more water soluble fibers comprises oligosaccharides.

In some embodiments, the one or more water soluble fibers comprises maltodextrin.

In some embodiments, the one or more water soluble fibers is maltodextrin.

In some embodiments, the one or more water soluble fibers comprises polydextrose.

In some embodiments, the one or more water soluble fibers is polydextrose.

In some embodiments, the one or more water soluble fibers is a mixture of two or more water soluble fibers.

In some embodiments, the one or more water soluble fibers provides cohesiveness and elasticity to the core. “Elasticity” is intended to be within the acceptable limits for “soft chew tablets” as described above. Hence, the texture may be greatly improved by use of the one or more water soluble fibers according to the invention. To the surprise of the inventors, it was possible to avoid using solid sugar syrups in the formulation by use of the one or more water soluble fibers according to the invention. The cohesiveness and elasticity may even be improved by avoiding the use of solid sugar syrups according to the invention. In the present context, “cohesiveness” or “coherent” is intended to mean the way the mass of the core behaves when it is for instance pulled apart. A high “cohesiveness” requires a higher force to pull the core apart. In the present context, “elasticity” is intended to mean the way the mass of the core behaves when it is for instance pulled apart. A high “elasticity” is understood in the same way as when a chewing gum is pulled apart, i.e., how the material bounces back upon chewing. In this context “elasticity” may sometimes also be referred to as “chewing resistance”.

In some embodiments, the one or more water soluble fibers provides reduced stickiness to the core. It was surprisingly seen that the one or more water soluble fibers may impact the stickiness of the core. This was not anticipated by the inventors, and greatly enhances the product, especially upon chewing the product, such as by sticking to the teeth during chewing. The formulation provided according to the invention serves to achieve this property.

In some embodiments, the one or more solid sugar syrups is present in an amount up to 15% by weight of the core. In some embodiments, the one or more solid sugar syrups is present in an amount up to 10% by weight of the core. In some embodiments, the one or more solid sugar syrups is present in an amount up to 8% by weight of the core. In some embodiments, the one or more solid sugar syrups is present in an amount up to 5% by weight of the core. In some embodiments, the one or more solid sugar syrups is present in an amount up to 3% by weight of the core. In some embodiments, the one or more solid sugar syrups is present in an amount up to 2% by weight of the core. In some embodiments, the one or more solid sugar syrups is present in an amount up to 1% by weight of the core.

In some embodiments, the one or more solid sugar syrups is present in a range of 0.01 to 20% by weight of the core. In some embodiments, the one or more solid sugar syrups is present in a range of 0.1 to 20% by weight of the core. In some embodiments, the one or more solid sugar syrups is present in a range of 1 to 20% by weight of the core. In some embodiments, the one or more solid sugar syrups is present in a range of 1 to 15% by weight of the core. In some embodiments, the one or more solid sugar syrups is present in a range of 1 to 10% by weight of the core. In some embodiments, the one or more solid sugar syrups is present in a range of 1 to 5% by weight of the core. In some embodiments, the one or more solid sugar syrups is present in a range of 1 to 3% by weight of the core. In some embodiments, the one or more solid sugar syrups is present in a range of 1 to 2% by weight of the core.

In some embodiments, the one or more solid sugar syrups is formed by first making a syrup, such as glucose syrup, followed by evaporation of water. In some embodiments, the one or more solid sugar syrups contain less than 6% by weight of water. “Solid sugar syrups” may sometimes also be referred to as “dried sugar syrups”

In some embodiments, the one or more solid sugar syrups has a Dextrose Equivalent (DE) of above 20. In some embodiments, the one or more solid sugar syrups has a Dextrose Equivalent (DE) of above 30.

In some embodiments, the one or more solid sugar syrups has a Dextrose Equivalent (DE) of 20 to 60. In some embodiments, the one or more solid sugar syrups has a Dextrose Equivalent (DE) of 30 to 50, In some embodiments, the one or more solid sugar syrups has a Dextrose Equivalent (DE) of 35 to 40,

In some embodiments, the one or more solid sugar syrups comprises one or more solid corn syrups. In some embodiments, the one or more solid sugar syrups comprises one or more solid glucose syrups.

In some embodiments, the one or more solid sugar syrups provides binding properties to the mass of the core.

In some embodiments, the one or more solid sugar syrups provides stickiness to the mass of the core. In some embodiments of the invention, stickiness is not preferred. In some other embodiments, stickiness is acceptable.

In some embodiments, the core does not comprise the one or more solid sugar syrups.

In some embodiments, the core comprises the one or more solid sugar syrups in a range of 0 to 20% by weight of the core. In some embodiments, the core comprises the one or more solid sugar syrups in a range of 0 to 10% by weight of the core. In some embodiments, the core comprises the one or more solid sugar syrups in a range of 0 to 5% by weight of the core. In some embodiments, the core comprises the one or more solid sugar syrups in a range of 0 to 3% by weight of the core. In some embodiments, the core comprises the one or more solid sugar syrups in a range of 0 to 2% by weight of the core. In some embodiments, the core comprises the one or more solid sugar syrups in a range of 0 to 1% by weight of the core.

In some embodiments, the core further comprises one or more liquid or semi-liquid sugar syrups.

In some embodiments, the core further comprises one or more liquid or semi-liquid sugar syrups in an amount of 0.1 to 30% by weight of the core. In some embodiments, the core further comprises one or more liquid or semi-liquid sugar syrups in an amount of 0.1 to 25% by weight of the core. In some embodiments, the core further comprises one or more liquid or semi-liquid sugar syrups in an amount of 0.1 to 20% by weight of the core. In some embodiments, the core further comprises one or more liquid or semi-liquid sugar syrups in an amount of 1 to 20% by weight of the core. In some embodiments, the core further comprises one or more liquid or semi-liquid sugar syrups in an amount of 1 to 10% by weight of the core.

In some embodiments, the core further comprises one or more liquid or semi-liquid sugar syrups providing binding properties and improved coherency to the mass of the core.

In some embodiments, the core further comprises one or more liquid or semi-liquid sugar syrups providing stickiness to the mass of the core. In some embodiments of the invention, stickiness is not preferred. In some other embodiments, stickiness is acceptable.

In some embodiments, the one or more powdered monosaccharides and/or disaccharides is present in an amount of 15 to 45% by weight of the core. In some embodiments, the one or more powdered monosaccharides and/or disaccharides is present in an amount of 15 to 40% by weight of the core. In some embodiments, the one or more powdered monosaccharides and/or disaccharides is present in an amount of 20 to 40% by weight of the core. In some embodiments, the one or more powdered monosaccharides and/or disaccharides is present in an amount of 20 to 40% by weight of the core. In some embodiments, the one or more powdered monosaccharides and/or disaccharides is present in an amount of 20 to 30% by weight of the core.

In some embodiments, the one or more powdered monosaccharides and/or disaccharides is one or more powdered monosaccharides.

In some embodiments, the one or more powdered monosaccharides is selected from the group consisting of glucose, fructose, galactose, and combinations thereof. In some embodiments, the one or more powdered monosaccharides is glucose. In some embodiments, the one or more powdered monosaccharides is fructose. In some embodiments, the one or more powdered monosaccharides is galactose.

In some embodiments, the one or more powdered monosaccharides and/or disaccharides is one or more powdered disaccharides. In some embodiments, the one or more powdered disaccharides is one or more non-reducing sugars.

In some embodiments, the one or more powdered disaccharides is selected from the group consisting of sucrose, trehalose, lactose, maltose, and combinations thereof. In some embodiments, the one or more powdered disaccharides is sucrose. In some embodiments, the one or more powdered disaccharides is trehalose. In some embodiments, the one or more powdered disaccharides is lactose. In some embodiments, the one or more powdered disaccharides is maltose.

In some embodiments, the one or more powdered monosaccharides and/or disaccharides is micro-pulverized.

In some embodiments, the one or more powdered monosaccharides and/or disaccharides has an average particle size of less than 100 microns, such as less than 75 microns, such as less than 50 microns. The average particles size being measured by standard laser diffraction techniques.

In some embodiments, the one or more powdered monosaccharides and/or disaccharides passes a screen cloth having a mesh opening of 0.074 mm (ASTM 200 mesh).

In some embodiments, the core further comprises inorganic mineral fillers. In some embodiments, the core further comprises calcium carbonate. In some embodiments, the core further comprises talc. In some embodiments, the core further comprises calcium carbonate in an amount of 5-20% by weight of the core. In some embodiments, the core further comprises calcium carbonate in an amount of 5-25% by weight of the core. In some embodiments, the core further comprises calcium carbonate in an amount of 5-30% by weight of the core. In some embodiments, the core further comprises calcium carbonate in an amount of 5-35% by weight of the core. In some embodiments, the core further comprises calcium carbonate in an amount of 10-20% by weight of the core. Addition of calcium carbonate is beneficial in order to provide an improved texture of the product.

In some embodiments, the core comprises water in an amount of no more than 8% by weight of the core before formation of the core, such as no more than 6% by weight. In some embodiments, the core comprises water in an amount of no more than 10% by weight of the core before formation of the core. In some embodiments, the core comprises water in an amount of no more than 6% by weight of the core before formation of the core.

In some embodiments, the core comprises unbound water in an amount of no more than 10% by weight of the core before formation of the core. In some embodiments, the core comprises unbound water in an amount of no more than 8% by weight of the core before formation of the core. In some embodiments, the core comprises unbound water in an amount of no more than 6% by weight of the core before formation of the core, such as no more than 4% by weight, such as no more than 2% by weight.

In some embodiments, the core comprises unbound water that provides binding properties to the core during formation.

In some embodiments, the core has a water activity of less than 0.6, such as less than 0.55, such as less than 0.5.

In some embodiments, the one or more texturizers is present in an amount of 0.5 to 15% by weight of the core. In some embodiments, the one or more texturizers is present in an amount of 1 to 12% by weight of the core. In some embodiments, the one or more texturizers is present in an amount of 1 to 15% by weight of the core. In some embodiments, the one or more texturizers is present in an amount of 5 to 15% by weight of the core. In some embodiments, the one or more texturizers is present in an amount of 10 to 15% by weight of the core. In some embodiments, the one or more texturizers is present in an amount of 2 to 10% by weight of the core. In some embodiments, the one or more texturizers is present in an amount of 1 to 10% by weight of the core. In some embodiments, the one or more texturizers is present in an amount of 0.5 to 8% by weight of the core. In some embodiments, the one or more texturizers is present in an amount of 1 to 5% by weight of the core.

In some embodiments, the one or more texturizers comprises one or more vegetable oils, such as hydrogenated vegetable oils. In the present context, “vegetable oil” is similar to “vegetable fat”.

In some embodiments, the one or more texturizers comprises one or more vegetable oils having a melting point below 40-50 degree Celsius.

In some embodiments, the one or more texturizers comprises one or more vegetable oils selected from the group consisting of coconut oil, palm oil, palm kernel oil, hydrogenated coconut oil, hydrogenated palm oil, hydrogenated palm kernel oil, and any combination thereof.

In some embodiments, the one or more texturizers comprises one or more medium chain triglycerides (MCT). In some embodiments, the one or more texturizers comprises one or more medium chain triglycerides (MCT) comprising C6-C12 triglycerides. In some embodiments, the one or more texturizers comprises one or more medium chain triglycerides (MCT) comprising C8-C10 triglycerides. In some embodiments, the one or more texturizers comprises one or more medium chain triglycerides (MCT) comprising capric acid. In some embodiments, the one or more texturizers comprises one or more medium chain triglycerides (MCT) comprising caprylic acid. In some embodiments, the one or more texturizers comprises one or more medium chain triglycerides (MCT) characterized by being liquid at 25 degrees Celsius.

In some embodiments, the one or more texturizers comprises one or more vegetable oils in an amount of 1 to 8% by weight of the core. In some embodiments, the one or more texturizers comprises one or more vegetable oils in an amount of 1 to 5% by weight of the core.

In some embodiments, the one or more texturizers comprises one or more vegetable oils in an amount of 2 to 4% by weight of the core.

In some embodiments, the one or more texturizers comprises one or more of lecithin, gelatine, acacia, or glycerin. In some embodiments, the one or more texturizers comprises lecithin. In some embodiments, the one or more texturizers comprises gelatine. In some embodiments, the one or more texturizers comprises acacia. In some embodiments, the one or more texturizers comprises glycerin. In some embodiments, the one or more texturizers comprises carrageenan. In some embodiments, the one or more texturizers comprises instant starch. Instant starch is also known as “pregelatinized starch”.

In some embodiments, the one or more texturizers comprises glycerin in an amount of 1 to 8% by weight of the core. In some embodiments, the one or more texturizers comprises glycerin in an amount of 1 to 5% by weight of the core. In some embodiments, the one or more texturizers comprises glycerin in an amount of 1 to 3% by weight of the core.

In some embodiments, the one or more texturizers comprises lecithin. In some embodiments, the one or more texturizers comprises gelatine. In some embodiments, the one or more texturizers comprises carrageenan. Gelatine with a higher bloom number is presently preferred. In some embodiments, the one or more texturizers comprises acacia. In some embodiments, the one or more texturizers comprises a vegetable oil, lecithin, gelatine, acacia, and glycerin. In some embodiments, the one or more texturizers comprises acacia. In some embodiments, the one or more texturizers comprises a vegetable oil, lecithin, carrageenan, acacia, and glycerin.

In some embodiments, the one or more texturizers comprises gelatine in an amount of no more than 5% by weight of the core. In some embodiments, the one or more texturizers comprises gelatine in an amount of no more than 10% by weight of the core.

In some embodiments, the one or more active ingredients is present in an amount of 10 to 60% by weight of the core. In some embodiments, the one or more active ingredients is present in an amount of 20 to 60% by weight of the core. In some embodiments, the one or more active ingredients is present in an amount of 20 to 50% by weight of the core. In some embodiments, the one or more active ingredients is present in an amount of 30 to 60% by weight of the core.

In some embodiments, the one or more active ingredients comprises one or more antacids.

In some embodiments, the one or more antacids is present in an amount of 20 to 60% by weight of the core. In some embodiments, the one or more antacids is present in an amount of 30 to 60% by weight of the core. In some embodiments, the one or more antacids is present in an amount of 40 to 60% by weight of the core. In some embodiments, the one or more antacids is present in an amount of 10 to 50% by weight of the core. In some embodiments, the one or more antacids is present in an amount of 20 to 50% by weight of the core. In some embodiments, the one or more antacids is present in an amount of 30 to 50% by weight of the core. In some embodiments, the one or more antacids is present in an amount of 25 to 45% by weight of the core.

In some embodiments, the one or more antacids is present in an amount of 1 to 50% by weight of the core. In some embodiments, the one or more antacids is present in an amount of 1 to 40% by weight of the core. In some embodiments, the one or more antacids is present in an amount of 1 to 30% by weight of the core. In some embodiments, the one or more antacids is present in an amount of 1 to 25% by weight of the core. In some embodiments, the one or more antacids is present in an amount of 1 to 20% by weight of the core. In some embodiments, the one or more antacids is present in an amount of 1 to 15% by weight of the core. In some embodiments, the one or more antacids is present in an amount of 1 to 5% by weight of the core. In some embodiments, the one or more antacids is present in an amount of 2 to 5% by weight of the core.

In some embodiments, the one or more antacids is selected from the group consisting of calcium carbonate, magnesium carbonate, sodium bicarbonate, magnesium hydroxide, aluminum hydroxide, magnesium silicate, simethicone, bismuth subsalicylate, and combinations thereof. Sodium alginate may be added in addition to calcium carbonate as well as other ingredients and actives.

In some embodiments, the one or more antacids comprises calcium carbonate. In some embodiments, the one or more antacids consists of calcium carbonate. In some embodiments, the one or more antacids comprises calcium carbonate in an amount of 20 to 60% by weight of the core. In some embodiments, the one or more antacids comprises calcium carbonate in an amount of 20 to 50% by weight of the core. In some embodiments, the one or more antacids comprises calcium carbonate in an amount of 30 to 60% by weight of the core. In some embodiments, the one or more antacids comprises calcium carbonate in an amount of 40 to 60% by weight of the core.

In some embodiments, the one or more active ingredients comprises one or more active ingredients with nutraceutical and/or pharmaceutical properties. In the present context, nutraceuticals is also known as dietary supplements.

In some embodiments, the one or more active ingredients comprises one or more lipophilic active ingredients.

In some embodiments, the one or more active ingredients comprises one or more electrolytic active ingredients. Electrolytic active ingredients include water soluble nutritive salts and may sometimes also be referred to as sports nutrition active ingredients.

In some embodiments, the one or more active ingredients comprises one or more energy stimulating active ingredients.

In some embodiments, the one or more active ingredients comprises one or more vitamins.

In some embodiments, the one or more active ingredients comprises one or more cannabinoids.

In some embodiments of the invention, the soft chew tablet comprises one or more cannabinoids. In some embodiments of the invention, the soft chew tablet comprises one or more cannabinoids in an amount of 1 to 300 mg. In some embodiments of the invention, the soft chew tablet comprises one or more cannabinoids in an amount of 1 to 200 mg. In some embodiments of the invention, the soft chew tablet comprises one or more cannabinoids in an amount of 1 to 100 mg. In some embodiments of the invention, the soft chew tablet comprises one or more cannabinoids in an amount of 2 to 50 mg. In some embodiments of the invention, the soft chew tablet comprises one or more cannabinoids in an amount of 5 to 30 mg. In some embodiments of the invention, the soft chew tablet comprises one or more cannabinoids in an amount of 10 to 20 mg. In some embodiments, any coating does not comprise one or more cannabinoids.

In some embodiments of the invention, the one or more cannabinoids is selected from the group consisting of cannabidiol (CBD), cannabidiolic acid (CBDA), tetrahydrocannabinol (THC), tetrahydrocannabinolic acid (THCA), cannabigerol (CBG), cannabichromene (CBC), cannabinol (CBN), cannabielsoin (CBE), iso-tetrahydrocannabinol (iso-THC), cannabicyclol (CBL), cannabicitran (CBT), cannabivarin (CBV), tetrahydrocannabivarin (THCV), cannabidivarin (CBDV), cannabichromevarin (CBCV), cannabigerovarin (CBGV), cannabigerol monomethyl ether (CBGM), and combinations thereof.

In some embodiments of the invention, the one or more cannabinoids is selected from the group consisting of cannabidiol (CBD), cannabidiolic acid (CBDA), cannabidivarin (CBDV), and combinations thereof.

In some embodiments of the invention, the one or more cannabinoids is selected from the group consisting of tetrahydrocannabinol (THC), tetrahydrocannabinolic acid (THCA), tetrahydrocannabivarin (THCV), and combinations thereof.

In some embodiments of the invention, the one or more cannabinoids comprises cannabidiol (CBD).

In some embodiments of the invention, the one or more cannabinoids comprises an isolated cannabinoid.

In some embodiments of the invention, the one or more cannabinoids is an isolated cannabinoid.

In some embodiments of the invention, the one or more cannabinoids is a synthetic cannabinoid.

In some embodiments of the invention, the one or more cannabinoids is not a cannabinoid extract with a cannabinoid purity of less than 80%.

In some embodiments, the one or more active ingredients comprises caffeine.

In some embodiments, the one or more active ingredients comprises L-theanine.

In some embodiments, the one or more active ingredients comprises collagen. In some embodiments, the one or more active ingredients comprises creatine. In some embodiments, the one or more active ingredients comprises BCAA. In some embodiments, the one or more active ingredients comprises milk proteins. In some embodiments, the one or more active ingredients comprises magnesium. In some embodiments, the one or more active ingredients comprises collagen. In some embodiments, the one or more active ingredients comprises hyaluronic acid. In some embodiments, the one or more active ingredients comprises keratin. In some embodiments, the one or more active ingredients comprises omega 3 fatty acids. In some embodiments, the one or more active ingredients comprises taurine.

In some embodiments, the one or more active ingredients does not comprise nicotine.

In some embodiments, the one or more active ingredients is present in an amount of 1 to 30% by weight of the core, such as 1 to 10% by weight of the core, such as 1 to 5% by weight of the core.

In some embodiments, the core has a hardness of more than 17 N (Newton) as measured on a standard texture analyzer known to one skilled in the art. A hardness of less than 17 N would not be preferable according to the invention. A certain hardness is typically required to allow for proper coating of the core of the present invention. Typically, the inventors by measuring the hardness of the soft chew tablets were able to distinguish inferior cores with cores fulfilling the requirement of the present invention in terms of texture.

In a fifth aspect, there is provided a soft chew tablet, the soft chew tablet comprising: a core constituting a coherent uncooked mass comprising: one or more soluble fibers in an amount of 10 to 45% by weight of the core, one or more powdered monosaccharides and/or disaccharides in an amount of 10 to 45% by weight of the core, one or more texturizers in an amount of 0.1 to 15% by weight of the core, one or more active ingredients in an amount of 1 to 60% by weight of the core, and one or more solid sugar syrups in an amount up to 20% by weight of the core.

The fifth aspect of the invention may include any one of the embodiments as described above. Preferably, the fifth aspect does not involve an outer coating. When a “core” is mentioned in the fifth aspect of the invention, reference is typically to be entire product, without a coating.

### DETAILED DESCRIPTION

The invention will now be described in more details with respect to certain aspects and embodiments of the invention. These aspects and embodiments are intended to be understood in connection with the rest of the description, including the Summary of the invention and the Examples of the invention.

As used herein, the term “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

As used herein, the term “%” and “percent” refers to percent by weight, unless otherwise is stated.

The verb “to comprise” as is used in this description and in the claims and its conjugations are used in its non-limiting sense to mean that items following the word are included, but items not specifically mentioned are not excluded. In addition, reference to an element by the indefinite article “a” or “an” does not exclude the possibility that more than one of the elements are present, unless the context clearly requires that there is one and only one of the elements. The indefinite article “a” or “an” thus usually means “at least one”. Additionally, the words “a” and “an” when used in the present document in connection with the word comprising or containing denote “one or more.” The expression “one or more” is intended to mean one, two, three or more.

The term “particle size” relates to the ability of the particles to move through or be retained by sieve holes of a specific size unless otherwise stated. As used herein, the term “particle size” refers to the average particle size as determined according to European Pharmacopoeia 9.1 when using test method 2.9.38 particle size distribution estimation by analytical sieving, unless otherwise specifically is mentioned.

The term “particle” or similar wording is intended to denote a single, discrete composition of solid matter, such as a granule or individual elements in powder, having a certain size that may deviate considerable.

By the phrase “texture” is meant a qualitative measure of the properties of the oral tablet and of the overall mouth-feel experienced by the user during use. Thus, the term “texture” encompasses measurable quantities such as hardness as well as more subjective parameters related to the feel experienced by a user.

The term “release” in the present context is intended to mean under “in vitro” conditions if not stated otherwise. In particular, the “release rate” during a certain period of time is intended to mean the amount in percentage of active ingredients that is released during the period. In the present context the term “release” refers to the released substance being liberated from the water-soluble matrix. In some embodiments, the process of releasing a substance corresponds to the substance being dissolved in saliva.

As used herein, the term “disintegrate” refers to a reduction of an object to components, fragments or particles. Disintegration time may be measured in vitro or in vivo. Unless otherwise stated, the in vitro measurements are carried out in accordance to European Pharmacopeia 9.0, section 2.9.1, Disintegration of tablets and capsules.

As used herein, the term “dissolve” is the process where a solid substance enters a solvent (oral saliva) to yield a solution. Unless otherwise stated, dissolving implies a full dissolving of the compound in question.

A “self-emulsifying agent” (as contained in SEDDS) is an agent which will form an emulsion when presented with an alternate phase with a minimum energy requirement. In contrast, an emulsifying agent, as opposed to a self-emulsifying agent, is one requiring additional energy to form an emulsion.

When referring to amounts of an ingredient by terms such as “less than”, “no more than”, this generally refers to the particular ingredient being absent or present in a range from trace amounts to the specified maximum amount.

As used herein the term “flavor” is understood as having its ordinary meaning within the art. Flavor includes liquid and powdered flavors. Thus, flavors do of course not include sweeteners (such as sugar, sugar alcohols and high intensity sweeteners), or acids providing pure acidity/sourness, nor compounds providing pure saltiness (e.g. NaCl) or pure bitterness. The flavors can be natural or synthetic flavors.

High intensity artificial sweetening agents may be applied according to the invention. For example, high intensity sweeteners include, but are not limited to sucralose, aspartame, salts of acesulfame, alitame, saccharin and its salts, cyclamic acid and its salts, glycyrrhizin, dihydrochalcones, thaumatin, monellin, stevioside (natural intensity sweetener) and the like, alone or in combination.

Usage level of the artificial sweetener will vary considerably and will depend on factors such as potency of the sweetener, rate of release, desired sweetness of the product, level and type of flavor used and cost considerations. Thus, the active level of artificial sweetener may vary from about 0.001 to about 8% by weight (such as from about 0.02 to about 8% by weight).

Usable flavors including as examples almond, almond amaretto, apple, Bavarian cream, black cherry, black sesame seed, blueberry, brown sugar, bubblegum, butterscotch, cappuccino, caramel, caramel cappuccino, cheesecake (graham crust), cinnamon redhots, cotton candy, circus cotton candy, clove, coconut, coffee, clear coffee, double chocolate, energy cow, graham cracker, grape juice, green apple, Hawaiian punch, honey, Jamaican rum, Kentucky bourbon, kiwi, koolada, lemon, lemon lime, tobacco, maple syrup, maraschino cherry, marshmallow, menthol, milk chocolate, mocha, Mountain Dew, peanut butter, pecan, peppermint, raspberry, banana, ripe banana, root beer, RY 4, spearmint, strawberry, sweet cream, sweet tarts, sweetener, toasted almond, tobacco, tobacco blend, vanilla bean ice cream, vanilla cupcake, vanilla swirl, vanillin, waffle, Belgian waffle, watermelon, whipped cream, white chocolate, wintergreen, amaretto, banana cream, black walnut, blackberry, butter, butter rum, cherry, chocolate hazelnut, cinnamon roll, cola, creme de menthe, eggnog, English toffee, guava, lemonade, licorice, maple, mint chocolate chip, orange cream, peach, pina colada, pineapple, plum, pomegranate, pralines and cream, red licorice, salt water taffy, strawberry banana, strawberry, kiwi, tropical punch, tutti frutti, vanilla, or any combination thereof.

In one embodiment the tablet according to the invention comprises a pharmaceutically, cosmetically or biologically active substance. Examples of such active substances, a comprehensive list of which is found e.g. in WO 00/25598, which is incorporated herein by reference, include drugs, dietary supplements, antiseptic agents, pH adjusting agents, anti-smoking agents and substances for the care or treatment of the oral cavity and the teeth such as hydrogen peroxide and compounds capable of releasing urea during chewing. Examples of useful active substances in the form of antiseptics include salts and derivatives of guanidine and biguanidine (for instance chlorhexidine diacetate) and the following types of substances with limited water-solubility: quaternary ammonium compounds (e.g. ceramine, chloroxylenol, crystal violet, chloramine), aldehydes (e.g. paraformaldehyde), derivatives of dequaline, polynoxyline, phenols (e.g. thymol, p-chlorophenol, cresol), hexachlorophene, salicylic anilide compounds, triclosan, halogenes (iodine, iodophores, chloroamine, dichlorocyanuric acid salts), alcohols (3,4 dichlorobenzyl alcohol, benzyl alcohol, phenoxyethanol, phenylethanol), cf. also Martindale, The Extra Pharmacopoeia, 28th edition, pages 547-578; metal salts, complexes and compounds with limited water-solubility, such as aluminum salts, (for instance aluminum potassium sulphate AlK(SO4)2, 12H2O) and salts, complexes and compounds of boron, barium, strontium, iron, calcium, zinc, (zinc acetate, zinc chloride, zinc gluconate), copper (copper chloride, copper sulphate), lead, silver, magnesium, sodium, potassium, lithium, molybdenum, vanadium should be included; other compositions for the care of mouth and teeth: for instance; salts, complexes and compounds containing fluorine (such as sodium fluoride, sodium monofluorophosphate, aminofluorides, stannous fluoride), phosphates, carbonates and selenium. Further active substances can be found in J. Dent. Res. Vol. 28 No. 2, pages 160-171, 1949.

Examples of active substances in the form of agents adjusting the pH in the oral cavity include: acids, such as adipic acid, succinic acid, fumaric acid, or salts thereof or salts of citric acid, tartaric acid, malic acid, acetic acid, lactic acid, phosphoric acid and glutaric acid and acceptable bases, such as carbonates, hydrogen carbonates, phosphates, sulphates or oxides of sodium, potassium, ammonium, magnesium or calcium, especially magnesium and calcium.

Active ingredients may comprise the below mentioned compounds or derivates thereof but are not limited thereto: Acetaminophen, Acetylsalicylic acid, Buprenorphine, Bromhexin, Celcoxib, Codeine, Diphenhydramin, Diclofenac, Etoricoxib, Ibuprofen, Indometacin, Ketoprofen, Lumiracoxib, Morphine, Naproxen, Oxycodon, Parecoxib, Piroxicam, Pseudoefedrin, Rofecoxib, Tenoxicam, Tramadol, Valdecoxib, Calciumcarbonat, Magaldrate, Disulfiram, Bupropion, Nicotine, Azithromycin, Clarithromycin, Clotrimazole, Erythromycin, Tetracycline, Granisetron, Ondansetrone, Prometazin, Tropisetron, Brompheniramine, Ceterizin, leco-Ceterizin, Chlorcyclizine, Chlorpheniramin, Chlorpheniramin, Difenhydramine, Doxylamine, Fenofenadin, Guaifenesin, Loratidin, des-Loratidin, Phenyltoloxamine, Promethazin, Pyridamine, Terfenadin, Troxerutin, Methyldopa, Methylphenidate, Benzalcon. Chloride, Benzeth. Chloride, Cetylpyrid. Chloride, Chlorhexidine, Ecabet-sodium, Haloperidol, Allopurinol, Colchinine, Theophylline, Propanolol, Prednisolone, Prednisone, Fluoride, Urea, Actot, Glibenclamide, Glipizide, Metformin, Miglitol, Repaglinide, Rosiglitazone, Apomorfin, Cialis, Sildenafil, Vardenafil, Diphenoxylate, Simethicone, Cimetidine, Famotidine, Ranitidine, Ratinidine, cetrizin, Loratadine, Aspirin, Benzocaine, Dextrometorphan, Phenylpropanolamine, Pseudoephedrine, Cisapride, Domperidone, Metoclopramide, Acyclovir, Dioctylsulfosucc., Phenolphtalein, Almotriptan, Eletriptan, Ergotamine, Migea, Naratriptan, Rizatriptan, Sumatriptan, Zolmitriptan, Aluminum salts, Calcium salts, Ferro salts, Ag-salts, Zinc-salts, Amphotericin B, Chlorhexidine, Miconazole, Triamcinolonacetonid, Melatonine, Phenobarbitol, Caffeine, Benzodiazepiner, Hydroxyzine, Meprobamate, Phenothiazine, Buclizine, Brometazine, Cinnarizine, Cyclizine, Difenhydramine, Dimenhydrinate, Buflomedil, Amphetamine, Caffeine, Ephedrine, Orlistat, Phenylephedrine, Phenylpropanolamin, Pseudoephedrine, Sibutramin, Ketoconazole, Nitroglycerin, Nystatin, Progesterone, Testosterone, Vitamin B12, Vitamin C, Vitamin A, Vitamin D, Vitamin E, Pilocarpin, Aluminumaminoacetat, Cimetidine, Esomeprazole, Famotidine, Lansoprazole, Magnesiumoxide, Nizatide and or Ratinidine.

The invention is suitable for increased or accelerated release of active agents selected among the group of dietary supplements, oral and dental compositions, antiseptic agents, pH adjusting agents, anti-smoking agents, sweeteners, flavorings, aroma agents or drugs. Some of those will be described below.

The active agents to be used in connection with the present invention may be any substance desired to be released from the tablet. The active agents, for which a controlled and/or accelerated rate of release is desired, are primarily substances with a limited water-solubility, typically below 10 g/100 mL inclusive of substances which are totally water-insoluble. Examples are medicines, dietary supplements, oral compositions, anti-smoking agents, highly potent sweeteners, pH adjusting agents, flavorings etc.

Other active ingredients are, for instance, paracetamol, benzocaine, cinnarizine, menthol, carvone, caffeine, chlorhexidine-di-acetate, cyclizine hydrochloride, 1,8-cineol, nandrolone, miconazole, mystatine, sodium fluoride, nicotine, cetylpyridinium chloride, other quaternary ammonium compounds, vitamin E, vitamin A, vitamin D, glibenclamide or derivatives thereof, progesterone, acetylsalicylic acid, dimenhydrinate, cyclizine, metronidazole, sodium hydrogen carbonate, the active components from ginkgo, the active components from propolis, the active components from ginseng, methadone, oil of peppermint, salicylamide, hydrocortisone or astemizole.

Examples of active agents in the form of dietary supplements are for instance salts and compounds having the nutritive effect of vitamin B2 (riboflavin), B12, folinic acid, folic acid, niacine, biotine, poorly soluble glycerophosphates, amino acids, the vitamins A, D, E and K, minerals in the form of salts, complexes and compounds containing calcium, phosphorus, magnesium, iron, zinc, copper, iodine, manganese, chromium, selenium, molybdenum, potassium, sodium or cobalt.

Furthermore, reference is made to lists of nutritionists accepted by the authorities in different countries such as for instance US code of Federal Regulations, Title 21, Section 182.5013.182 5997 and 182.8013-182.8997.

Examples of active agents in the form of antiseptics are for instance salts and compounds of guanidine and biguanidine (for instance chlorhexidine diacetate) and the following types of substances with limited water-solubility: quaternary ammonium compounds (for instance ceramine, chloroxylenol, crystal violet, chloramine), aldehydes (for instance paraformaldehyde), compounds of dequaline, polynoxyline, phenols (for instance thymol, para chlorophenol, cresol) hexachlorophene, salicylic anilide compounds, triclosan, halogenes (iodine, iodophores, chloroamine, dichlorocyanuric acid salts), alcohols (3,4 dichlorobenzyl alcohol, benzyl alcohol, phenoxyethanol, phenylethanol), cf. furthermore Martindale, The Extra Pharmacopoeia, 28th edition, pages 547-578; metal salts, complexes and compounds with limited water-solubility, such as aluminum salts, (for instance aluminum potassium sulphate AlK(SO4)2,12H2O) and furthermore salts, complexes and compounds of boron, barium, strontium, iron, calcium, zinc, (zinc acetate, zinc chloride, zinc gluconate), copper (copper chloride, copper sulfate), lead, silver, magnesium, sodium, potassium, lithium, molybdenum, vanadium should be included; other compositions for the care of mouth and teeth: for instance; salts, complexes and compounds containing fluorine (such as sodium fluoride, sodiummonofluorophosphate, amino fluorides, stannous fluoride), phosphates, carbonates and selenium.

Cf. furthermore J. Dent. Res. Vol. 28 No. 2, pages 160-171, 1949, wherein a wide range of tested compounds is mentioned.

Examples of active agents in the form of agents adjusting the pH in the oral cavity include for instance: acceptable acids, such as adipic acid, succinic acid, fumaric acid, or salts thereof or salts of citric acid, tartaric acid, malic acid, acetic acid, lactic acid, phosphoric acid and glutaric acid and acceptable bases, such as carbonates, hydrogen carbonates, phosphates, sulfates or oxides of sodium, potassium, ammonium, magnesium or calcium, especially magnesium and calcium.

Examples of active agents in the form of anti-smoking agents include for instance: nicotine, tobacco powder or silver salts, for instance silver acetate, silver carbonate and silver nitrate.

Further examples of active agents are medicines of any type.

Examples of active agents in the form of medicines include caffeine, salicylic acid, salicyl amide and related substances (acetylsalicylic acid, choline salicylate, magnesium salicylate, sodium salicylate), paracetamol, salts of pentazocine (pentazocine hydrochloride and pentazocinelactate), buprenorphine hydrochloride, codeine hydrochloride and codeine phosphate, morphine and morphine salts (hydrochloride, sulfate, tartrate), methadone hydrochloride, ketobemidone and salts of ketobemidone (hydrochloride), beta-blockers, (propranolol), calcium antagonists, verapamil hydrochloride, nifedinpine as well as suitable substances and salts thereof mentioned in Pharm. Int., November 85, pages 267-271, Barney H. Hunter and Robert L. Talbert, nitroglycerine, erythrityl tetranitrate, strychnine and salts thereof, lidocaine, tetracaine hydrochloride, etorphine hydrochloride, atropine, insulin, enzymes (for instance papain, trypsin, amyloglucosidase, glucoseoxidase, streptokinase, streptodomase, dextranase, alpha amylase), polypeptides (oxytocin, gonadorelin, (LH.RH), desmopressin acetate (DDAVP), isoxsuprine hydrochloride, ergotamine compounds, chloroquine (phosphate, sulfate), isosorbide, demoxytocin, heparin.

Other active ingredients include beta-lupeol, Letigen®, Sildenafil citrate and derivatives thereof.

Further examples of active ingredients include dental products including Carbamide, CPP Caseine Phospho Peptide; Chlorhexidine, Chlorhexidine di acetate, Chlorhexidine Chloride, Chlorhexidine di gluconate, Hexetedine, Strontium chloride, Potassium Chloride, Sodium bicarbonate, Sodium carbonate, Fluor containing ingredients, Fluorides, Sodium fluoride, Aluminum fluoride.

Further examples of active ingredients include Ammonium fluoride, Calcium fluoride, Stannous fluoride, Other fluor containing ingredients Ammonium fluorosilicate, Potassium fluorosilicate, Sodium fluorosilicate, Ammonium monofluorphosphate, Calcium monofluorphosphate, Potassium monofluorphosphate, Sodium monofluorphosphate, Octadecentyl Ammonium fluoride, Stearyl Trihydroxyethyl Propylenediamine Dihydrofluoride

Further examples of active ingredients include vitamins. Vitamins include A, B1, B2, B6, B12, Folinic acid, Folic acid, niacin, Pantothenic acid, biotine, C, D, E, K. Minerals include Calcium, phosphor, magnesium, iron, Zinc, Copper, Iod, Mangan, Crom, Selene, Molybden. Other active ingredients include:

Q10®, enzymes. Natural drugs including Ginkgo Biloba, ginger, and fish oil.

Further examples of active ingredients include migraine drugs such as Serotonin antagonists: Sumatriptan, Zolmitriptan, Naratriptan, Rizatriptan, Eletriptan; nausea drugs such as Cyclizin, Cinnarizin, Dimenhydramin, Difenhydrinat; hay fever drugs such as Cetrizin, Loratidin, pain relief drugs such as Buprenorfin, Tramadol, oral disease drugs such as Miconazol, Amphotericin B, Triamcinolonaceton; and the drugs Cisaprid, Domperidon, Metoclopramid. In a preferred embodiment the invention relates to the release of Nicotine and its salts.

In an advantageous embodiment of the invention the active ingredient is selected from active ingredients for the throat selected from acetylcysteine, ambroxol, amylmetacresol, benzocaine, bisacodyl, bismuth subsalicylate, bromhexine, cetirizine, cetylpyridinium, chlorhexidine, dextromethorphan hydrobromide, 2,4-dichlorobenzyl alcohol, doxylamine succinate, eucalyptus oil, flurbiprofen, glycerin, hexylresorcinol, lidocaine, menthol, myrrh, paracetamol, pectin, peppermint oil, phenol, phenylephrine, povidone-iodine, pseudoephedrine, ranitidine, simethicone, sodium docusate, spearmint, zinc, or any combination thereof; active ingredients for the gastrointestinal tract selected from alginate, atenolol, aspirin (acetylsalicylic acid), ampicillin, aminosalicylates, anhydrous citric acid, aspirin, bisacodyl, bismuth subsalicylate, bupropion, caffeine, calcium, calcium carbonate, cetirizine, cimetidine, cisapride, clarithromycin, desloratadine, dexlansoprazole, diphenhydramine HCl, diphenhydramine citrate, dimenhydrinate, docusate erythromycin, dopamine, esomeprazole, famotidine, fexofenadine HCl, guaifenesin, hydrotalcite, ibuprofen, ketoprofen, lactase enzyme, lansoprazole, loratadine, lorcaserin, loperamide, loperamide HCl, magnesium, magnesium carbonate, magnesium hydroxide, melatonin, methamphetamine HCl, metoclopramide, metronidazole, montelukast, mycostatin, naltrexone, naproxen, naproxen sodium, nizatidine, omeprazole, ondansetron, orlistat, pantoprazole, paracetamol(acetaminophen), pectin, phentermine HCl, polypodium leucotomos, prednisolone, prednisone, progesterone, propranolol, propantheline bromide, pseudoephedrine HCl, phentermine, rabeprazole, ranitidine, roflumilast, scopoloamine butyl hydroxide, simethicone, sodium, sodium bicarbonate, sodium docusate, sumatriptan, testosterone, tetracycline, topiramate, vitamin A, vitamin B, vitamin B12, vitamin C (ascorbic acid), vitamin D, and vitamin E, vitamin K, or any combination thereof, and active ingredients for buccal absorption selected from atenolol, baclofen, caffeine, carvedilol, chlorpheniramine, chlorpheniramine maleate, fluticasone propionate, maleate, desmopressin, diltiazem hydrochloride, doxylamine succinate, mycostatin, nicotine, nifedipine, nitroglycerin, omeprazole, ondansetron, oxymetazoline HCl, oxytocin, phenylephrine, piroxicam, prednisone, propranolol, salbutamol sulphate, scopoloamine butyl hydroxide, sumatriptan, triamcinolonacetonid, and any combination thereof.

According to the invention, the one or more cannabinoids may be selected from various cannabinoids.

“Cannabinoids” are a group of compounds including the endocannabinoids, the phytocannabinoids and those which are neither endocannabinoids or phytocannabinoids, hereinafter “syntho-cannabinoids”.

“Endocannabinoids” are endogenous cannabinoids, which may have high affinity ligands of CB1 and CB2 receptors.

“Phytocannabinoids” are cannabinoids that originate in nature and can be found in the cannabis plant. The phytocannabinoids can be present in an extract including a botanical drug substance, isolated, or reproduced synthetically.

“Syntho-cannabinoids” are those compounds capable of interacting with the cannabinoid receptors (CB1 and/or CB2) but are not found endogenously or in the cannabis plant. Examples include WIN 55212 and rimonabant.

An “isolated phytocannabinoid” is one which has been extracted from the cannabis plant and purified to such an extent that the additional components such as secondary and minor cannabinoids and the non-cannabinoid fraction have been substantially removed.

A “synthetic cannabinoid” is one which has been produced by chemical synthesis. This term includes modifying an isolated phytocannabinoid, by, for example, forming a pharmaceutically acceptable salt thereof.

A “substantially pure” cannabinoid is defined as a cannabinoid which is present at greater than 95% (w/w) pure. More preferably greater than 96% (w/w) through 97% (w/w) thorough 98% (w/w) to 99% % (w/w) and greater.

A “highly purified” cannabinoid is defined as a cannabinoid that has been extracted from the cannabis plant and purified to the extent that other cannabinoids and non-cannabinoid components that are co-extracted with the cannabinoids have been substantially removed, such that the highly purified cannabinoid is greater than or equal to 95% (w/w) pure.

“Plant material” is defined as a plant or plant part (e.g. bark, wood, leaves, stems, roots, flowers, fruits, seeds, berries or parts thereof) as well as exudates, and includes material falling within the definition of “botanical raw material” in the Guidance for Industry Botanical Drug Products Draft Guidance, August 2000, US Department of Health and Human Services, Food and Drug Administration Center for Drug Evaluation and Research.

In the context of this application the terms “cannabinoid extract” or “extract of cannabinoids”, which are used interchangeably, encompass “Botanical Drug Substances” derived from cannabis plant material. A Botanical Drug Substance is defined in the Guidance for Industry Botanical Drug Products Draft Guidance, August 2000, US Department of Health and Human Services, Food and Drug Administration Centre for Drug Evaluation and Research as: “A drug substance derived from one or more plants, algae, or macroscopic fungi. It is prepared from botanical raw materials by one or more of the following processes: pulverisation, decoction, expression, aqueous extraction, ethanolic extraction, or other similar processes.” A botanical drug substance does not include a highly purified or chemically modified substance derived from natural sources. Thus, in the case of cannabis, “botanical drug substances” derived from cannabis plants do not include highly purified, Pharmacopoeial grade cannabinoids.

The term “Cannabis plant(s)” encompasses wild type Cannabis sativa and also variants thereof, including cannabis chemovars which naturally contain different amounts of the individual cannabinoids, Cannabis sativa subspecies indica including the variants var. indica and var. kafiristanica, Cannabis indica, Cannabis ruderalis and also plants which are the result of genetic crosses, self-crosses or hybrids thereof. The term “Cannabis plant material” is to be interpreted accordingly as encompassing plant material derived from one or more cannabis plants. For the avoidance of doubt it is hereby stated that “cannabis plant material” includes dried cannabis biomass.

Preferably the one or more cannabinoids are selected from: cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBO), tetrahydrocannabinol (THC), tetrahydrocannabinolic acid (THCA), tetrahydrocannabivarin (THCV) and tetrahydrocannabivarinic acid (THCV A). More preferably the one or more cannabinoid is CBD or THC. This list is not exhaustive and merely details the cannabinoids which are identified in the present application for reference.

So far, more than 120 different phytocannabinoids have been identified which are within the scope of the present invention.

Cannabinoids can be split into different groups as follows: Phytocannabinoids; Endocannabinoids; and Synthetic cannabinoids.

Cannabinoid receptors can be activated by three major groups of agonist ligands, for the purposes of the present invention and whether or not explicitly denominated as such herein, lipophilic in nature and classed respectively as: endocannabinoids (produced endogenously by mammalian cells); phytocannabinoids (such as cannabidiol, produced by the cannabis plant); and, synthetic cannabinoids (such as HU-210).

Phytocannabinoids can be found as either the neutral carboxylic acid form or the decarboxylated form depending on the method used to extract the cannabinoids. For example, it is known that heating the carboxylic acid form will cause most of the carboxylic acid form to decarboxylate.

Phytocannabinoids can also occur as either the pentyl (5 carbon atoms) or propyl (3 carbon atoms) variant. For example, the phytocannabinoid THC is known to be a CB1 receptor agonist whereas the propyl variant THCV has been discovered to be a CB1 receptor antagonist meaning that it has almost opposite effects.

According to the invention, examples of phytocannabinoids may be cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBO), tetrahydrocannabinol (THC), tetrahydrocannabinolic acid (THCA), tetrahydrocannabivarin (THCV) and tetrahydrocannabivarinic acid (THCV A). More preferably the one or more cannabinoid is CBD or THC.

The formulation according to the present invention may also comprise at least one cannabinoid selected from those disclosed in A. Douglas Kinghorn et al., Phytocannabinoids, Vol. 103, Chapter 1, pages 1-30.

Examples of endocannabinoids are molecules that activate the cannabinoid receptors within the body. Examples include 2-arachidonyl glycerol (2AG), 2-arachidonyl glyceryl ether (2AGE), arachidonyl dopamine, and arachidonyl ethanolamide (anandamide). Structurally related endogenous molecules have been identified that share similar structural features, but that display weak or no activity towards the cannabinoid receptors but are also termed endocannabinoids. Examples of these endocannabinoid lipids include 2-acyl glycerols, alkyl or alkenyl glyceryl ethers, acyl dopamines and N-acylethanolamides that contain alternative fatty acid or alcohol moieties, as well as other fatty acid amides containing different head groups. These include N-acylserines as well as many other N-acylated amino acids.

Examples of cannabinoid receptor agonists are neuromodulatory and affect short-term memory, appetite, stress response, anxiety, immune function and analgesia.

In one embodiment the cannabinoid is palmitoylethanolamide (PEA) which is an endogenous fatty acid amide belonging to the class of nuclear factor agonists.

Synthetic cannabinoids encompass a variety of distinct chemical classes: the cannabinoids structurally related to THC, the cannabinoids not related to THC, such as (cannabimimetics) including the aminoalkylindoles, 1,5-diarylpyrazoles, quinolines, and arylsulfonamides, and eicosanoids related to the endocannabinoids. All or any of these cannabinoids can be used in the present invention.

It is preferred that the formulation comprises one or two primary cannabinoids, which are preferably selected from the group consisting of, cannabidiol (CBD) or cannabidivarin (CBDV), tetrahydrocannabinol (THC), tetrahydrocannabivarin (THCV), tetrahydrocannabinolic acid (THCA), cannabigerol (CBG) and cannabidiolic acid (CBDA) or a combination thereof. It is preferred that the formulation comprises cannabidiol and/or tetrahydrocannabinol.

The following non-limiting examples illustrate different variations of the present invention. The examples are meant for indicating the inventive concept; hence the mentioned examples should not be understood as exhaustive for the present.

### EXAMPLES

### Example 1

### Preparation of Soft Chew Cores

Compositions were prepared in a process of low speed shear mixing. The mixing process was conducted in a 2000 g Sigma blade kneader mixer operated with a speed of 40-50 rpm and a mixing temperature of 45-50 degree Celsius.

Before processing, powdered mono- or disaccharides was pulverized to a particle size below 100 microns by grinding. The powdered mono- or disaccharide was both supplied in a grade with particles up to 0.074 mm, or grinded and passed through a screen having a mesh opening of 0.074 mm (200 mesh ASTM).

One or more water soluble fibers was premixed with the powdered one or more mono- or disaccharides in the low speed shear mixer for about 2 minutes, whereafter one or more texturizers was added including vegetable oil and lecithin emulsifier (if any) and mixed for about 3 minutes. Further texturizers (if any) including one or more of acacia, gelatine, carrageenan, and glycerin were then added and mixed for about 2 minutes. Hereafter, further ingredients were added, such as flavor, color, and one or more active ingredients, and the mixture was blended until a total blending time of 10-12 minutes. If water was added, this was done after about 5 minutes of mixing, and in that case acacia and gelatine (if any) might preferably be premixed with the water at elevated temperature, whereas if one or more solid sugar syrups was added, this was done together with the mono- or disaccharides at the beginning of the mixing process, ie. at time 0. If one or more liquid or semi-liquid sugar syrups was added, this was done at the same time as further texturizers, i.e., after about 5 minutes of mixing.

The mass of the inventive examples had a consistency of a soft chew tablet mass with visual appearance of an unspun coherent mass that was easy to transfer to other equipment for further processing into desired shapes.

The soft chew tablet mass obtained by low shear mixing (individual batches) was cooled to a temperature of about 20-30 degree Celsius and formed into ropes by extrusion followed by cutting and forming into round-shaped soft chew cores of about 2-2.5 g per piece by means of a rotary forming machine. From a side view, the cores had an ellipse form. The temperature of cooling was critical in order to avoid problems in the formation of the soft chew tablet cores.

### Example 2

### Formulation of Coated Soft Chew Tablets

Soft chew cores prepared according to Example 1 were coated with a hard coating after storage for about 12-24 hours.

A suspension containing aqueous crystallizable sugar was applied onto the cores in a standard pan coating process, in which the water contents of the suspension applied was removed by blowing with air. This cycle was repeated 40-80 times in order to reach the swelling required, i.e., an increase in weight or thickness of the product.

A sub-coat was optionally applied around the soft chew cores in a pan coating process prior to applying the hard coating as outlined above. The pre-coat applied, if any, was performed with a blend in a specified ratio of Capol® 236 N provided by Capol GmbH, a brand of Freudenberg and powdered sucrose. The weight of the sub-coat was about 0.25-2% by weight of the soft chew tablet.

The hard coating consisted of 65% by weight of sucrose crystals, 4% by weight of acacia, 30% by weight of unbound water and about 1% color and flavor. The total weight of the hard coating was about 10-30% by weight of the soft chew tablet, the final weight of the hard coating depending directly on the number of coating cycles applied.

### Example 3

### Formulation of Soft Chew Cores

Soft chew tablet cores with a weight of 2.2 g each were prepared according to Example 1.

TABLE 1

| | B1 | B2 | B3 | B4 | B5 | B6 | B7 | B8 | C1 |
|---|---|---|---|---|---|---|---|---|---|
| Calcium carbonate | 750 | 750 | 750 | 750 | 750 | 750 | 750 | 750 | 750 |
| [mg/tablet] | | | | | | | | | |
| Powdered sucrose | 27.8 | 28.2 | 30.8 | 27.0 | 26.8 | 24.2 | 5.0 | 24.2 | 0 |
| Powdered dextrose | 0 | 0 | 0 | 0 | 0 | 15 | 5 | 0 | 42 |
| Maltodextrin | 25 | 25 | 25 | 25 | 25 | 10 | 40 | 25 | 0 |
| Glucose syrup | 0 | 0 | 0 | 0 | 0 | 0 | 0 | 6 | 23 |
| Vegetable oil | 4 | 4 | 4 | 1 | 7 | 4 | 4 | 4 | 0 |
| Emulsifier | 0.3 | 0.3 | 0.3 | 0 | 0.3 | 0.3 | 0 | 0.3 | 0 |
| Gelatine | 1.8 | 1.8 | 1.8 | 2.7 | 1.8 | 1.8 | 1.3 | 1.8 | 0 |
| Acacia | 0.4 | 0 | 0.4 | 0.6 | 0.4 | 1 | 1 | 1 | 0 |
| Water | 6 | 3 | 0 | 6 | 0 | 6 | 6 | 0 | 0 |
| Glycerin | 0 | 3 | 3 | 3 | 4 | 3 | 3 | 3 | 0.3 |
| Flavors | 0.7 | 0.7 | 0.7 | 0.7 | 0.7 | 0.7 | 0.7 | 0.7 | 0.7 |
| Calcium carbonate | 34 | 34 | 34 | 34 | 34 | 34 | 34 | 34 | 34 |
| Total | 100 | 100 | 100 | 100 | 100 | 100 | 100 | 100 | 100 |

Raw material contents in %. Calcium carbonate grade Omyapure 35-OG was obtained from Omya. Powdered sucrose and powdered dextrose was prepared according to Example 1. Maltodextrin was applied as Maltodextrin C*Dry ™ MD 01915 from Cargill

Soft chew tablet cores with a weight of 2.2 g each were prepared according to Example 1.

TABLE 2

| | B11 | B12 | B13 | B14 | B15 | B16 | B17 | B18 |
|---|---|---|---|---|---|---|---|---|
| Calcium | 750 | 750 | 750 | 750 | 750 | 750 | 750 | 750 |
| carbonate | | | | | | | | |
| [mg/tablet] | | | | | | | | |
| Powdered | 17.8 | 24.8 | 23.7 | 22.8 | 26.3 | 24.2 | 10 | 12.3 |
| sucrose | | | | | | | | |
| Maltodextrin | 25 | 25 | 25 | 25 | 25 | 25 | 20 | 25 |
| Solid sugar | 7 | 0 | 0 | 0 | 0 | 0 | 19.8 | 12.5 |
| syrup | | | | | | | | |
| Vegetable oil | 4 | 4 | 4 | 4 | 2.7 | 4 | 4 | 4 |
| Emulsifier | 0.3 | 0.3 | 0.3 | 0.3 | 0.1 | 0.3 | 0.3 | 0.3 |
| Gelatine | 1.8 | 1.8 | 2.7 | 1.8 | 1.8 | 1.8 | 1.8 | 1.8 |
| Acacia | 0.4 | 0.4 | 0.6 | 0.4 | 0.4 | 1 | 0.4 | 0.4 |
| Water | 6 | 6 | 6 | 6 | 6 | 6 | 6 | 6 |
| Glycerin | 3 | 3 | 3 | 5 | 3 | 3 | 3 | 3 |
| Flavors | 0.7 | 0.7 | 0.7 | 0.7 | 0.7 | 0.7 | 0.7 | 0.7 |
| Calcium | 34 | 34 | 34 | 34 | 34 | 34 | 34 | 34 |
| carbonate | | | | | | | | |
| Total | 100 | 100 | 100 | 100 | 100 | 100 | 100 | 100 |

Raw material contents in %. Calcium carbonate grade Omyapure 35-OG was obtained from Omya. Powdered sucrose was prepared according to Example 1. Solid sugar syrup was applied as Dried Glucose Syrup C*Dry ™ GL 01934 from Cargill. Maltodextrin was applied as Maltodextrin C*Dry ™ MD 01915 from Cargill

Soft chew tablet cores with a weight of 2.2 g each were prepared according to Example 1.

TABLE 3

| | B20 | B21 | B22 | B23 | B24 | B25 | B26 | B27 |
|---|---|---|---|---|---|---|---|---|
| Calcium | 100 | 250 | 250 | 500 | 500 | 1000 | 1000 | 1250 |
| carbonate | | | | | | | | |
| [mg/tablet] | | | | | | | | |
| Powdered | 38.7 | 35.5 | 35.5 | 30.3 | 30.3 | 18.7 | 18.7 | 12.8 |
| sucrose | | | | | | | | |
| Maltodextrin | 40 | 36.3 | 36.3 | 31.2 | 31.2 | 19 | 19 | 13.6 |
| Solid sugar | 0 | 0 | 2 | 0 | 2 | 0 | 2 | 0 |
| syrup | | | | | | | | |
| Vegetable oil | 4 | 4 | 2 | 4 | 2 | 4 | 2 | 4 |
| Emulsifier | 0.3 | 0.3 | 0.3 | 0.3 | 0.3 | 0.3 | 0.3 | 0.3 |
| Gelatine | 1.8 | 1.8 | 1.8 | 1.8 | 1.8 | 1.8 | 1.8 | 1.8 |
| Acacia | 1 | 1 | 1 | 1 | 1 | 1 | 1 | 1 |
| Water | 6 | 6 | 6 | 6 | 6 | 6 | 6 | 6 |
| Glycerin | 3 | 3 | 3 | 3 | 3 | 3 | 3 | 3 |
| Flavors | 0.7 | 0.7 | 0.7 | 0.7 | 0.7 | 0.7 | 0.7 | 0.7 |
| Calcium | 4.5 | 11.4 | 11.4 | 22.7 | 22.7 | 45.5 | 45.5 | 56.8 |
| carbonate | | | | | | | | |
| Total | 100 | 100 | 100 | 100 | 100 | 100 | 100 | 100 |

Raw material contents in %. Calcium carbonate grade Omyapure 35-OG was obtained from Omya. Powdered sucrose was prepared according to Example 1. Solid sugar syrup was applied as Dried Glucose Syrup C*Dry ™ GL 01934 from Cargill. Maltodextrin was applied as Maltodextrin C*Dry ™ MD 01915 from Cargill

Soft chew tablet cores with a weight of 2.2 g each were prepared according to Example 1.

TABLE 3A

| | B27A | B27B | B27C | B27D | B27E | B27F | B27G | B27H |
|---|---|---|---|---|---|---|---|---|
| Vitamin C | 100 | 250 | 250 | 500 | 500 | 1000 | 1000 | 125 |
| [mg/tablet] | | | | | | | | |
| Powdered | 38.7 | 35.4 | 27.4 | 30.3 | 21.3 | 18.9 | 10.4 | 21.3 |
| sucrose | | | | | | | | |
| Maltodextrin | 40 | 35.4 | 27.4 | 30.2 | 21.2 | 18.8 | 10.3 | 21.2 |
| Calcium | 0 | 0 | 17 | 0 | 17 | 0 | 17 | 34 |
| carbonate | | | | | | | | |
| Vegetable oil | 4 | 4 | 4 | 4 | 4 | 4 | 4 | 4 |
| Emulsifier | 0.3 | 0.3 | 0.3 | 0.3 | 0.3 | 0.3 | 0.3 | 0.3 |
| Gelatine | 1.8 | 1.8 | 1.8 | 1.8 | 1.8 | 1.8 | 1.8 | 1.8 |
| Acacia | 1 | 1 | 1 | 1 | 1 | 1 | 1 | 1 |
| Water | 6 | 6 | 6 | 6 | 6 | 6 | 6 | 6 |
| Glycerin | 3 | 3 | 3 | 3 | 3 | 3 | 3 | 3 |
| Flavors | 0.7 | 0.7 | 0.7 | 0.7 | 0.7 | 0.7 | 0.7 | 0.7 |
| Vitamin C | 4.5 | 11.4 | 11.4 | 22.7 | 22.7 | 45.5 | 45.5 | 5.7 |
| Vitamin D | 0 | 1.0 | 0 | 0 | 1.0 | 0 | 0 | 1.0 |
| Total | 100 | 100 | 100 | 100 | 100 | 100 | 100 | 100 |

Raw material contents in %. Calcium carbonate grade Omyapure 35-OG was obtained from Omya. Powdered sucrose was prepared according to Example 1. Maltodextrin was applied as Maltodextrin C*Dry ™ MD 01915 from Cargill. Vitamin C was applied as Ascorbic Acid Fine Powder from DSM. Vitamin D was applied as Dry Vitamin D3 100 SD/S from DSM with a load of vitamin D of 2.5 mcg/mg (mcg = microgram).

Soft chew tablet cores with a weight of 2.2 g each were prepared according to Example 1.

TABLE 4

| | B30 | B31 | B32 | B33 | B34 | B35 | B36 | B37 |
|---|---|---|---|---|---|---|---|---|
| Calcium | 750 | 750 | 750 | 750 | 750 | 750 | 500 | 500 |
| carbonate | | | | | | | | |
| [mg/tablet] | | | | | | | | |
| Powdered | 39.2 | 39.2 | 29.2 | 29.2 | 19.2 | 19.2 | 15.2 | 15.2 |
| sucrose | | | | | | | | |
| Maltodextrin | 10 | 10 | 20 | 20 | 30 | 30 | 45 | 45 |
| Solid sugar | 0 | 2 | 0 | 2 | 0 | 2 | 0 | 2 |
| syrup | | | | | | | | |
| Vegetable oil | 4 | 2 | 4 | 2 | 4 | 2 | 4 | 2 |
| Emulsifier | 0.3 | 0.3 | 0.3 | 0.3 | 0.3 | 0.3 | 0.3 | 0.3 |
| Gelatine | 1.8 | 1.8 | 1.8 | 1.8 | 1.8 | 1.8 | 1.8 | 1.8 |
| Acacia | 1 | 1 | 1 | 1 | 1 | 1 | 1 | 1 |
| Water | 6 | 6 | 6 | 6 | 6 | 6 | 6 | 6 |
| Glycerin | 3 | 3 | 3 | 3 | 3 | 3 | 3 | 3 |
| Flavors | 0.7 | 0.7 | 0.7 | 0.7 | 0.7 | 0.7 | 0.7 | 0.7 |
| Calcium | 34 | 34 | 34 | 34 | 34 | 34 | 23 | 23 |
| carbonate | | | | | | | | |
| Total | 100 | 100 | 100 | 100 | 100 | 100 | 100 | 100 |

Raw material contents in %. Calcium carbonate grade Omyapure 35-OG was obtained from Omya. Powdered sucrose was prepared according to Example 1. Solid sugar syrup was applied as Dried Glucose Syrup C*Dry ™ GL 01934 from Cargill. Maltodextrin was applied as Maltodextrin C*Dry ™M MD 01915 from Cargill.

Soft chew tablet cores with a weight of 2.2 g each were prepared according to Example 1.

TABLE 4A

| | B37A | B37B | B37C | B37D | B37E | B37F | B37G | B37H |
|---|---|---|---|---|---|---|---|---|
| Vitamin D | 55 | 55 | 55 | 55 | 55 | 55 | 55 | 55 |
| [mcg/tablet] | | | | | | | | |
| Powdered | 32.9 | 24.4 | 24.4 | 15.4 | 41.5 | 23.4 | 24.2 | 24.4 |
| sucrose | | | | | | | | |
| Maltodextrin | 32.9 | 24.4 | 24.4 | 15.4 | 41.5 | 23.4 | 24.2 | 24.4 |
| Vegetable oil | 4 | 4 | 4 | 4 | 4 | 4 | 4 | 4 |
| Emulsifier | 0.5 | 0.5 | 0.5 | 0.5 | 0.5 | 0.5 | 0.5 | 0.5 |
| Gelatine | 2 | 2 | 2 | 0 | 2 | 0 | 0.9 | 0 |
| Carrageenan | 0 | 0 | 0 | 0 | 0 | 0 | 0 | 2 |
| Instant Starch | 0 | 0 | 0 | 20 | 0 | 0 | 0 | 0 |
| Acacia | 0.5 | 0.5 | 0.5 | 0.5 | 0.5 | 4.5 | 2 | 0.5 |
| Water | 6 | 6 | 6 | 6 | 6 | 6 | 6 | 6 |
| Glycerin | 3 | 3 | 3 | 3 | 3 | 3 | 3 | 3 |
| Flavors | 0.2 | 0.2 | 0.2 | 0.2 | 0 | 0.2 | 0.2 | 0.2 |
| Calcium | 17 | 34 | 0 | 34 | 0 | 34 | 34 | 34 |
| carbonate | | | | | | | | |
| Talc | 0 | 0 | 34 | 0 | 0 | 0 | 0 | 0 |
| Vitamin D | 1.0 | 1.0 | 1.0 | 1.0 | 1.0 | 1.0 | 1.0 | 1.0 |
| Total | 100 | 100 | 100 | 100 | 100 | 100 | 100 | 100 |

Raw material contents in %. Calcium carbonate grade Omyapure 35-OG was obtained from Omya. Powdered sucrose was prepared according to Example 1. Maltodextrin was applied as Maltodextrin C*Dry ™ MD 01915 from Cargill. Vitamin D was applied as Dry Vitamin D3 100 SD/S from DSM with a load of vitamin D of 2.5 mcg/mg (mcg = microgram).

Soft chew tablet cores with a weight of 2.0 g each were prepared according to Example 1.

TABLE 5

| | B40 | B41 | B42 | B43 | B44 | B45 | B46 | B47 |
|---|---|---|---|---|---|---|---|---|
| Calcium | 750 | 750 | 750 | 750 | 750 | 750 | 750 | 750 |
| carbonate | | | | | | | | |
| [mg/tablet] | | | | | | | | |
| Powdered | 17.8 | 24.8 | 23.7 | 22.8 | 26.3 | 24.2 | 10 | 12.3 |
| sucrose | | | | | | | | |
| Polydextrose | 25 | 25 | 25 | 25 | 25 | 25 | 20 | 25 |
| Solid sugar | 7 | 0 | 0 | 0 | 0 | 0 | 19.8 | 12.5 |
| syrup | | | | | | | | |
| Vegetable oil | 4 | 4 | 4 | 4 | 2.7 | 4 | 4 | 4 |
| Emulsifier | 0.3 | 0.3 | 0.3 | 0.3 | 0.1 | 0.3 | 0.3 | 0.3 |
| Gelatine | 1.8 | 1.8 | 2.7 | 1.8 | 1.8 | 1.8 | 1.8 | 1.8 |
| Acacia | 0.4 | 0.4 | 0.6 | 0.4 | 0.4 | 1 | 0.4 | 0.4 |
| Water | 6 | 6 | 6 | 6 | 6 | 6 | 6 | 6 |
| Glycerin | 3 | 3 | 3 | 5 | 3 | 3 | 3 | 3 |
| Flavors | 0.7 | 0.7 | 0.7 | 0.7 | 0.7 | 0.7 | 0.7 | 0.7 |
| Calcium | 34 | 34 | 34 | 34 | 34 | 34 | 34 | 34 |
| carbonate | | | | | | | | |
| Total | 100 | 100 | 100 | 100 | 100 | 100 | 100 | 100 |

Raw material contents in %. Calcium carbonate grade Omyapure 35-OG was obtained from Omya. Powdered sucrose was prepared according to Example 1. Solid sugar syrup was applied as Dried Glucose Syrup C*Dry ™ GL 01934 from Cargill.

Soft chew tablet cores with a weight of 2.0 g each were prepared according to Example 1.

TABLE 6

| | B50 | B51 | B52 | B53 | B54 | B55 | B56 | B57 |
|---|---|---|---|---|---|---|---|---|
| Caffeine | 40 | 80 | 40 | 80 | 40 | 80 | 40 | 80 |
| [mg/tablet] | | | | | | | | |
| Powdered | 33.7 | 38.7 | 39.6 | 36.7 | 42.2 | 38.1 | 825.9 | 26.2 |
| sucrose | | | | | | | | |
| Maltodextrin | 41.1 | 41.1 | 41.1 | 41.1 | 41.1 | 41.1 | 36.1 | 41.1 |
| Solid sugar | 7 | 0 | 0 | 0 | 0 | 0 | 19.8 | 12.5 |
| syrup | | | | | | | | |
| Vegetable oil | 4 | 4 | 4 | 4 | 2.7 | 4 | 4 | 4 |
| Emulsifier | 0.3 | 0.3 | 0.3 | 0.3 | 0.1 | 0.3 | 0.3 | 0.3 |
| Gelatine | 1.8 | 1.8 | 2.7 | 1.8 | 1.8 | 1.8 | 1.8 | 1.8 |
| Acacia | 0.4 | 0.4 | 0.6 | 0.4 | 0.4 | 1 | 0.4 | 0.4 |
| Water | 6 | 6 | 6 | 6 | 6 | 6 | 6 | 6 |
| Glycerin | 3 | 3 | 3 | 5 | 3 | 3 | 3 | 3 |
| Flavors | 0.7 | 0.7 | 0.7 | 0.7 | 0.7 | 0.7 | 0.7 | 0.7 |
| Caffeine | 2.0 | 4.0 | 2.0 | 4.0 | 2.0 | 4.0 | 2.0 | 4.0 |
| Total | 100 | 100 | 100 | 100 | 100 | 100 | 100 | 100 |

Raw material contents in %. Caffeine grade was “Natural Caffeine Powder” obtained from CR3, Bremen, Germany. Powdered sucrose was prepared according to Example 1. Solid sugar syrup was applied as Dried Glucose Syrup C*Dry ™ GL 01934 from Cargill.

Soft chew tablet cores with a weight of 2.2 g each were prepared according to Example 1.

TABLE 7

| | B60 | B61 | B62 | B63 | B64 | B65 | B66 | B67 |
|---|---|---|---|---|---|---|---|---|
| Calcium | 750 | 750 | 750 | 750 | 750 | 750 | 750 | 750 |
| carbonate | | | | | | | | |
| [mg/tablet] | | | | | | | | |
| Powdered | 24.2 | 24.2 | 24.2 | 24.2 | 24.2 | 24.2 | 24.2 | 24.2 |
| sucrose | | | | | | | | |
| Maltodextrin | 25 | 25 | 25 | 25 | 25 | 25 | 25 | 25 |
| Solid sugar | 0 | 0 | 0 | 0 | 0 | 0 | 0 | 0 |
| syrup | | | | | | | | |
| Vegetable oil | 0 | 3 | 6 | 9 | 0 | 3 | 6 | 9 |
| Emulsifier | 0.3 | 0.3 | 0.3 | 0.3 | 0.3 | 0.3 | 0.3 | 0.3 |
| Gelatine | 1.7 | 1.7 | 1.7 | 1.7 | 1.7 | 1.7 | 1.7 | 1.7 |
| Acacia | 0 | 0 | 0 | 0 | 1 | 1 | 1 | 1 |
| Water | 6 | 6 | 6 | 6 | 6 | 6 | 6 | 6 |
| Glycerin | 3 | 3 | 3 | 3 | 3 | 3 | 3 | 3 |
| Flavors | 0.7 | 0.7 | 0.7 | 0.7 | 0.7 | 0.7 | 0.7 | 0.7 |
| Calcium | 34 | 34 | 34 | 34 | 34 | 34 | 34 | 34 |
| carbonate | | | | | | | | |
| Total | 100 | 100 | 100 | 100 | 100 | 100 | 100 | 100 |

Raw material contents in %. Calcium carbonate grade Omyapure 35-OG was obtained from Omya. Powdered sucrose was prepared according to Example 1. Solid syrup powder was applied as Dried Glucose Syrup C*Dry ™ GL 01934 from Cargill.

Relevant for all inventive samples above, the cores that were provided with a hard coating according to example 2 revealed surprisingly advantageous properties as a partial moisture barrier to ingredients in the core of the soft chew tablet.

Additionally, relevant for all inventive samples above, the cores that were provided with a hard coating revealed surprisingly advantageous properties as a partial anti-sticking barrier to ingredients in the core of the soft chew tablet. The sub-coat further increased the moisture barrier properties as well as the anti-sticking barrier properties.

It is noted that calcium carbonate in the formulations above may be applied as an active ingredient. However, calcium carbonate may also be applied in the formulations above to improve the texture of the soft chew tablets in addition to the one or more texturizers. Hence, calcium carbonate may have two functions as a special case. Calcium carbonate is not included in the definition of one or more texturizers according to the invention.

### Example 4

### Sensorial Evaluation of Coated Soft Chew Tablets

The soft chew cores of Example 3 were evaluated after application of coating according to Example 2.

For evaluation a panel of 7 trained assessors was used. Each assessor repeated the evaluations twice.

The trained assessors abstain from eating and drinking at least 30 minutes before initiation of any test. Each trained assessor was a healthy person appointed on an objective basis according to specified requirements. The sensory analysis was performed according to ISO 4121-2003 in testing conditions following ISO 8589. The result is an average of the results of the 7 individuals.

The test persons gave a rating from “+” to “+++++”, where “+” is poor and “+++++” is excellent.

Rating “+++” means that the tablet was comparable to the standard, whereas “+” means that the tablet was very far from comparable to the standard and “+++++” means that the tablet is exceeding the rating of the standard. “0” indicated that it was not tested.

Different parameters were tested in a test panel:

| | | | |
|---|---|---|---|
| Mouthfeel | Tackiness | Softness | Off-notes |

“Mouthfeel”—the general impression of the tablet when placed in the mouth with respect to elements such as cohesiveness, elasticity, and texture.

“Tackiness” the ability of the tablet to stick to teeth during chewing. A soft chew tablet should possess some tackiness but neither too little or too much. I.e. a score of “+++” is considered ideal for this parameter

“Softness”—the softness of the tablet. Both a slightly softer and a slightly harder than average tablet would be considered a good soft chew tablet

“Off-notes”—the overall impression of the off-note from the one or more active ingredients in the composition during chewing. For instance, if off-notes (chalkiness, bitter notes, irritation in the throat) were experienced, a low rating was given and if other uncomfortable sensations were experienced, a low rating was also given.

### Example 5

### Qualitative Sensorial Evaluation of Coated Soft Chew Tablets

A qualitative sensorial evaluation was made of the soft chew cores of Example 3 after application of coating according to Example 2. The coating cycles were repeated until a weight gain of 27% was obtained for each samples.

| | Total sensory | |
|---|---|---|
| | experience: | |
| | Very good | |
| | Good | |
| | Acceptable(Acc) | |
| | Poor | |
| Sample | Very poor | Comments |
| C1 | Very poor | Soft chewing texture, very little chewing |
| | | resistance, poor tackiness |
| B12 | Very good | Slightly firm chewing texture, very good |
| | | chewing resistance, good “just right” |
| | | tackiness |
| B16 | Very good | Soft chewing texture, very good chewing |
| | | resistance, good “just right” tackiness |
| B17 | Good | Soft chewing texture, good chewing |
| | | resistance, slightly high tackiness |
| B18 | Good | Slightly firm chewing |
| | | texture, good chewing |
| | | resistance, slightly high tackiness |
| Ref | Good | Alka-Seltzer calcium carbonate antacid |
| | | chewable tablets |
| | | Soft chewing texture, good chewing |
| | | resistance, good “just right” tackiness |

### Example 6

### Hardness Tests

Various samples of representative soft chew cores were tested on a standard texture analyzer in order to measure the hardness of selected soft chew cores before a coating was applied according to Example 2 was provided. The texture analyzer was provided by Stable Micro Systems under the name TAXT2i Texture Analyser. The texture analyzer was equipped with a needle that was applied to the top surface of the cores. The force applied to penetrate the cores was measured in Newton (N).

TABLE 8

| Hardness (N) | B37A | B37B | B37C | B37D | B37E | B37F | B37G | B37H |
|---|---|---|---|---|---|---|---|---|
| 2 days | 13 | 19 | 19 | 18 | 33 | 13 | 12 | 15 |
| 6 days | 20 | 26 | 25 | 34 | 43 | 17 | 18 | 19 |
| 10 days | 26 | 31 | 32 | 44 | 52 | 21 | 23 | 23 |

Measured on standard texturizer during storage over time.

As can be seen from the tests of hardness, various of the samples had a superior hardness already after a few days of storage, whereas the hardness was reached for other samples after more days. All these samples fall within the desired hardness according to the invention. When the same samples were applied with an outer hard coating shortly after reaching a hardness of 17 N, it was seen that the hardness of the cores was maintained at about the same level.

One of the advantages of the present invention is that stability of active ingredients may be surprisingly improved, such as the stability for vitamins. Compared to other products, such as gummies and products made by cooking, the soft chew tablet of the present invention was seen to offer better stability to active ingredients. Also, compared to tablets with a higher water content, the instant soft chew tablets may be improved in terms of stability. Improvement on stability was particularly seen for sample B27H compared to gummies but was also tested for other samples.

1-103. (canceled)

104. A coated soft chew tablet, the soft chew tablet comprising:
a core in an amount of 60 to 99% by weight of the soft chew tablet, the core constituting a coherent uncooked mass comprising:
one or more water soluble fibers in an amount of 10 to 45% by weight of the core,
one or more powdered monosaccharides and/or disaccharides in an amount of 10 to 45% by weight of the core,
one or more texturizers in an amount of 0.1 to 15% by weight of the core,
one or more active ingredients in an amount of 1 to 60% by weight of the core, and
one or more solid sugar syrups in an amount up to 20% by weight of the core; and
an outer coating surrounding the core in an amount of 1 to 40% by weight of the soft chew tablet.

105. The coated soft chew tablet according to claim 104, wherein the outer coating comprises sugar in an amount of 30 to 98% by weight of the coating.

106. The coated soft chew tablet according to claim 104, wherein the outer coating is a hard coating.

107. The coated soft chew tablet according to claim 104, wherein the outer coating is a coating selected from the group consisting of a film coating, a soft coating, and a chocolate coating.

108. The coated soft chew tablet according to claim 104, wherein the soft chew tablet further comprises a sub-coat applied between the core and the outer coating.

109. The coated soft chew tablet according to claim 104, wherein the coherent uncooked mass has not been re-crystallized during formation of the core.

110. The coated soft chew tablet according to claim 104, wherein the coherent uncooked mass is characterized by preserved crystallinity of the one or more powdered monosaccharides and/or disaccharides during formation of the core.

111. The coated soft chew tablet according to claim 104, wherein the one or more water soluble fibers has a Dextrose Equivalent (DE) of 3 to 20.

112. The coated soft chew tablet according to claim 104, wherein the one or more water soluble fibers comprises oligosaccharides.

113. The coated soft chew tablet according to claim 104, wherein the core further comprises one or more liquid or semi-liquid sugar syrups in an amount of 0.1 to 30% by weight of the core.

114. The coated soft chew tablet according to claim 104, wherein the one or more powdered monosaccharides and/or disaccharides has an average particle size of less than 100 microns.

115. The coated soft chew tablet according to claim 104, wherein the core further comprises calcium carbonate in an amount of 5-20% by weight of the core.

116. The coated soft chew tablet according to claim 104, wherein the core has a water activity of less than 0.6.

117. The coated soft chew tablet according to claim 104, wherein the one or more texturizers comprises one or more vegetable oils having a melting point below 40-50 degree Celsius.

118. The coated soft chew tablet according to claim 104, wherein the one or more texturizers comprises one or more medium chain triglycerides (MCT).

119. The coated soft chew tablet according to claim 104, wherein the one or more texturizers comprises one or more of lecithin, gelatine, carrageenan, instant starch, acacia, or glycerin.

120. The coated soft chew tablet according to claim 104, wherein the one or more texturizers comprises a vegetable oil, lecithin, gelatine, acacia, and glycerin.

121. The coated soft chew tablet according to claim 104, wherein the one or more active ingredients is present in an amount of 10 to 60% by weight of the core.

122. The coated soft chew tablet according to claim 104, wherein the one or more active ingredients comprises one or more antacids.

123. The coated soft chew tablet according to claim 104, wherein the one or more active ingredients comprises one or more antacids selected from the group consisting of calcium carbonate, magnesium carbonate, sodium bicarbonate, magnesium hydroxide, aluminum hydroxide, magnesium silicate, simethicone, bismuth subsalicylate, and combinations thereof.

124. The coated soft chew tablet according to claim 104, wherein the one or more active ingredients comprises one or more active ingredients with nutraceutical and/or pharmaceutical properties.

125. The coated soft chew tablet according to claim 104, wherein the one or more active ingredients is present in an amount of 1 to 30% by weight of the core.

126. A soft chew tablet, the soft chew tablet comprising:
a core constituting a coherent uncooked mass comprising:
one or more water soluble fibers in an amount of 10 to 45% by weight of the core,
one or more powdered monosaccharides and/or disaccharides in an amount of 10 to 45% by weight of the core,
one or more texturizers in an amount of 0.1 to 15% by weight of the core,
one or more active ingredients in an amount of 1 to 60% by weight of the core, and
one or more solid sugar syrups in an amount up to 20% by weight of the core.
